(19) United States
(12) Patent Application Publication
(10) Pub. No.: US 2022/0130539 A1
(43) Pub. Date: Apr. 28, 2022

(54) METHOD AND SYSTEM FOR DYNAMICALLY GENERATING PROFILE-SPECIFIC THERAPEUTIC IMAGERY USING MACHINE LEARNING MODELS

(71) Applicant: Mahana Therapeutics, Inc., San Francisco, CA (US)

(72) Inventor: Simon Levy, San Francisco, CA (US)

(73) Assignee: Mahana Therapeutics, Inc., San Francisco, CA (US)

(21) Appl. No.: 17/217,052

(22) Filed: Mar. 30, 2021

Related U.S. Application Data
(60) Provisional application No. 63/104,322, filed on Oct. 22, 2020.

(51) Int. Cl. G16H 50/20; G16H 30/40; G16H 50/50

(52) U.S. Cl. CPC G16H 50/20; G16H 50/50; G16H 30/40

(57) ABSTRACT

Therapeutic imagery is selected for administration to a patient, and the therapy is administered to the patient. The patient's responses to the therapeutic imagery are monitored, collected, and correlated with the associated therapeutic imagery attribute data to generate data indicating the effectiveness of the imagery. The therapeutic imagery effectiveness data is used as training data to train one or more machine learning based therapeutic imagery effectiveness prediction models. Data associated with one or more new therapeutic imagery attributes is provided as input to one or more of the trained therapeutic imagery effectiveness prediction models, which generates predicted therapeutic imagery effectiveness data for the new therapeutic imagery. The therapeutic imagery effectiveness data is analyzed to determine and select one or more effective therapeutic imagery attributes, resulting in generation of maximally effective therapeutic imagery.

### RELATED APPLICATIONS

This application claims the benefit of Paull et al, U.S. Provisional Patent Application No. 63/104,322, filed on Oct. 22, 2020, entitled “SYSTEMS AND METHODS FOR GUIDED ADMINISTRATION OF BEHAVIORAL THERAPY,” which is hereby incorporated by reference in its entirety as if it were fully set forth herein.

This application is related to U.S. patent application Ser. No. ______ (attorney docket number MAH008), naming Simon Levy as inventor, filed concurrently with the present application on Mar. 30, 2021, entitled “METHOD AND SYSTEM FOR DYNAMICALLY GENERATING PROFILE-SPECIFIC THERAPEUTIC PROTOCOLS USING MACHINE LEARNING MODELS,” which is hereby incorporated by reference in its entirety as if it were fully set forth herein. This application is also related to U.S. patent application Ser. No. ______ (attorney docket number MAH009), naming Simon Levy as inventor, filed concurrently with the present application on Mar. 30, 2021, entitled “METHOD AND SYSTEM FOR DYNAMICALLY GENERATING GENERALIZED THERAPEUTIC PROTOCOLS USING MACHINE LEARNING MODELS,” which is hereby incorporated by reference in its entirety as if it were fully set forth herein. This application is also related to U.S. patent application Ser. No. ______ (attorney docket number MAH011), naming Simon Levy as inventor, filed concurrently with the present application on Mar. 30, 2021, entitled “METHOD AND SYSTEM FOR DYNAMICALLY GENERATING GENERALIZED THERAPEUTIC IMAGERY USING MACHINE LEARNING MODELS,” which is hereby incorporated by reference in its entirety as if it were fully set forth herein.

### BACKGROUND

Every day, millions of people are diagnosed with a wide variety of medical conditions, ranging greatly in type and severity. A patient who has been diagnosed with a medical condition often experiences many hardships as a result of their diagnosis. In addition to physical effects, such as pain, discomfort, or loss of mobility that may accompany the diagnosis, the hardships faced by patients often further include financial difficulties resulting from lost work, medical bills and the cost of treatments. Further still, a patient's diagnosis often negatively impacts their social interactions, quality of life, and overall emotional well-being. The result is that many patients experience significant physical and psychological distress, and often do not receive effective support or treatment to alleviate this distress.

Additionally, psychological distress often exacerbates the physical symptoms associated with a patient's diagnosis, which in turn can lead to even greater psychological distress. As one specific example, symptoms associated with a gastrointestinal (GI) disorder, such as irritable bowel syndrome (IBS), are often triggered by stress, and psychological issues such as depression and/or anxiety can worsen those symptoms. Often, when a patient is diagnosed with one or more medical conditions, the patient is referred to additional health practitioners for further care and treatment. For example, a patient who has been diagnosed with a gastrointestinal (GI) disorder may be referred to a psychologist, psychiatrist, counselor, or other mental health practitioner to address any psychological issues, such as stress, anxiety, and/or depression that may stem from the diagnosis. Health practitioners such as these typically utilize one or more techniques, methodologies, and/or modalities such as, but not limited to, cognitive behavioral therapy (CBT), acceptance commitment therapy (ACT), dialectical behavioral therapy (DBT), exposure therapy, hypnotherapy, experiential therapy, and psychodynamic therapy, to assist patients with management of their physiological and/or psychological conditions.

Many of these types of therapies fall under a broad category referred to as psychotherapy. Psychotherapy methodologies can be utilized to help patients manage a wide range of psychological and physical conditions. One tool often utilized by health practitioners, typically as part of one or more therapy sessions, is that of therapeutic imagery, which may also be referred to as guided imagery, guided meditation, or visualization. Therapeutic imagery is often classified as a “mind-body intervention,” which refers to one or more practices that employ a variety of techniques designed to facilitate the mind's capacity to affect bodily function and systems. There are many documented scientific studies that show benefits of mind-body interventions in contributing to the treatment of a wide range of conditions including but not limited to, chronic pain, stress, anxiety, and depression.

Therapeutic imagery may be administered to a patient as a stand-alone treatment, or may be incorporated into a particular type of therapy, such as CBT. Even though a behavioral therapy, such as CBT, can be implemented in a variety of ways, it is typically administered utilizing a set of structured sessions or modules, which are each designed to teach a patient skills that enable the patient to better understand and manage thoughts and behaviors that may be negatively affecting their mental and physical states. Although aspects of CBT may be fairly structured, there is still enough flexibility to allow CBT to be adapted for use in treatment of particular conditions. One such example is that of utilizing CBT to treat IBS. The rationale for applying CBT to treat IBS is grounded in the biopsychosocial model, which states that one's biology, thoughts, emotions, external events, and behaviors influence IBS symptom expression in a bidirectional way.

Thus, utilizing mind-body intervention techniques, such as therapeutic imagery, in one or more sessions or modules of a behavioral therapy, such as CBT, can be effective in treating a wide variety of conditions, such as IBS. Although the specific examples of CBT and IBS are referenced throughout the present specification, it should be noted that applications of therapeutic imagery may extend to many other types of conditions and therapies for treating those conditions. Additionally, although therapeutic imagery is often administered to patients by a health practitioner in clinical settings, therapeutic imagery may also be administered to participants in non-clinical settings. Therapeutic imagery may be administered to patients or participants across a range of delivery modalities. For example, the therapeutic imagery may be administered by a health practitioner in-person, individually, or in-person, in a group. Alternatively, the therapeutic imagery may be administered remotely, such as telephonically, over the internet, or through a computer software application. Further, a therapy may be administered to a patient without the direct involvement of a health practitioner, for example, a therapy may be administered to a patient remotely by a software application and/or may be self-administered by the patient.

Typically a trained practitioner uses therapeutic imagery to help a patient or participant generate mental images that simulate sensory perceptions and/or elicit strong emotions or feelings. Therapeutic imagery may encompass textual descriptions and/or visual images of places, objects, activities, colors, shapes, textures, sounds, smells and/or any other sensory experiences. Therapeutic imagery may be administered to a patient or participant via any type of audiovisual media, including but not limited to, spoken text, still images, video recordings, recordings of music, speech, and/or sound, and/or interactive media, such as through therapeutic software and virtual reality.

Traditionally, in clinical settings, a health practitioner may determine that a patient would benefit from the use of therapeutic imagery as part of a therapeutic treatment, and so the health practitioner may select one or more therapeutic imagery scenes, scripts, or techniques to administer to a patient. However, there are currently a limited number of therapeutic imagery scenes, scripts, or techniques available that have been clinically validated, or that have been proven to be effective for use in therapy. Additionally, therapeutic imagery that is effective for one patient may not be effective for a different patient, and further, therapeutic imagery that is effective for one patient on one day, or at one moment in time, may not be effective for the same patient on a different day, or at a different moment in time.

There are various ways to determine effectiveness of therapeutic imagery. For example, upon administration of therapeutic imagery to a patient, data may be solicited, generated and/or collected regarding the effectiveness of the therapeutic imagery. Certain types of therapeutic imagery may be found to be effective for the average patient population, for particular types of patients, or for one particular individual patient. Further, therapeutic imagery that is effective for one particular type of condition, such as stress, may not be effective for a different type of condition, such as chronic pain. Upon a determination that traditional, or historical therapeutic imagery is not effective for its intended recipient, the therapeutic imagery may be adjusted, or new therapeutic imagery may be selected in an effort to find therapeutic imagery that is more effective for its intended recipient.

Unfortunately, this process traditionally involves a lot of guesswork and/or trial and error on the part of the health practitioner, which results in delayed, ineffective, and/or counterproductive treatment for patients, who may become unhappy, discouraged, frustrated, and/or, may lose faith in the therapeutic process. Clearly, finding a way to quickly and efficiently determine whether particular therapeutic imagery will be effective for patients can greatly assist the health practitioner in their ability to provide effective care, support, and treatment to patients. Currently, there are a small number of rudimentary measures that may be taken to identify therapeutic imagery that will be effective for a patient, such as simply asking the patient to select from a number of predefined, static scenes (e.g. “tropical beach scene,” or “wooded forest scene,” etc.)

Unfortunately, due to the sheer quantity of variables involved in creating therapeutically effective imagery, the problem rapidly becomes a monumental technical task, one which cannot readily be accomplished by the human mind, even with pen and paper, and even given unlimited time. For example, health data related to thousands of health conditions and symptoms associated with those conditions must be analyzed, and the effectiveness of millions of potential imagery attributes must also be analyzed and correlated with the health data, thousands of metrics and correlations between those metrics may be involved in determining which imagery is therapeutically effective and which is not. The problem is further compounded when you take into account that while certain imagery may be therapeutically effective for one type of patient, the same imagery may not be therapeutically effective for other types of patients, and so the attributes of the therapeutic imagery need to be tailored to the characteristics of particular patients in order to be maximally effective. Further, there is currently no solution to the technical problem of determining how to dynamically adjust therapeutic imagery to maximize therapeutic effectiveness. Therapeutic imagery is traditionally static, using predefined scripts, as well as predefined audio and imagery, and cannot be easily modified dynamically. In order to be maximally effective, therapeutic imagery should be reactive to changes in a patient's moods, emotions, and physiological state and be able to dynamically adjust to the patient's needs in real-time.

Therefore, generation of effective therapeutic imagery presents a technical problem, which requires a technical solution. As software applications continue to replace human interactions, this problem becomes even more pronounced, as people are increasingly relying on applications to provide them with support and assistance in a wide variety of aspects of their daily lives. This is especially true in times of global crises, such as the 2020 worldwide pandemic, which has limited the availability and/or desirability of in-person medical appointments. When administering therapeutic imagery remotely, for example, over the internet, through a website, or through a software application, the imagery utilized is traditionally statically programmed into the software and thus is not able to be readily modified when new data is received, such as data relating to the state of the participant and/or the effectiveness of the imagery. Thus, due to the large number of people with psychological and/or physical difficulties, as well as the increasing demand for remote administration of therapies, the failure of traditional solutions to address the problem of dynamically generating therapeutic imagery in order to quickly and efficiently administer effective therapies to patients/participants, has the potential to lead to significant consequences for a large number of people.

What is needed, therefore, is a technical solution to the technical problem of dynamically generating therapeutic imagery to ensure that patients/participants receive effective care, support, and treatment.

### SUMMARY

Embodiments of the present disclosure provide a technical solution to the technical problem of dynamically generating therapeutic imagery to ensure that patients/participants receive effective care, support, and treatment. In one embodiment, when a patient has been diagnosed with one or more health conditions, an appropriate therapy may be selected for administration to the patient, depending on the patient's particular diagnosis. In many instances, the therapy selected may be a psychological therapy that is intended to treat psychological issues related to the patient's diagnosis. As used herein the term “therapy,” “psychological therapy,” or “therapeutic modality,” may include psychological techniques, methodologies, and/or modalities utilized to treat patients, such as, but not limited to psychotherapy, mind-body intervention, cognitive behavioral therapy (CBT), acceptance commitment therapy (ACT), dialectical behavioral therapy (DBT), exposure therapy, hypnotherapy, experiential therapy, and psychodynamic therapy.

In one embodiment, part of the selected therapy may include administering therapeutic imagery to a patient. In one embodiment, therapeutic imagery may be administered to a patient as a stand-alone treatment outside of the structure of a particular therapy. In one embodiment, therapeutic imagery may be administered or provided to a participant who has not necessarily been diagnosed with a health condition, outside of a clinical setting. Therefore, although the term “patient” will be used commonly throughout the enclosed specification, the term “participant” may also be used to indicate that applications of the methods and systems disclosed herein, used outside of a clinical setting, are also contemplated by the following disclosure.

In one embodiment, historical therapeutic imagery is selected to administer or provide to a patient. As used herein, the phrase “administration of a therapy” may include administration of a therapy to a patient by a health practitioner, or administration of a therapy to a patient without the direct involvement of a health practitioner. As used herein, the term “historical therapeutic imagery” may include therapeutic imagery that has previously been generated, tested, established, and/or clinically validated for use in administration to patients.

In one embodiment, the historical therapeutic imagery is administered or provided to the patient via one or more delivery modalities. For example, the historical therapeutic imagery may be administered by a health practitioner in-person, individually, or in-person, in a group. Alternatively, the historical therapeutic imagery may be administered remotely, such as telephonically, over the internet, or through a computer software application. Further, a therapy may be administered to a patient without the direct involvement of a health practitioner, for example, a therapy may be administered to a patient remotely by a software application and/or may be self-administered by the patient. In various embodiments, therapeutic imagery may be text-based, audio-based, image-based, video-based, virtual reality-based or may comprise any combination of multimedia elements.

In one embodiment, as the historical therapeutic imagery is being administered to the patient, the patient's responses to the historical therapeutic imagery are monitored to obtain patient imagery response data. Patient imagery response data may also be collected after administration of the therapeutic imagery. As used herein, in various embodiments, “patient response data” or “patient imagery response data” may include direct verbal or written feedback from the patient, indirect feedback, such as an indication of whether particular therapeutic imagery appears to be having an effect on the patient, and/or other measureable data such as, but not limited to, physiological sensor data, and/or click-stream data showing patient engagement with the therapeutic imagery.

In one embodiment, the patient imagery response data is analyzed to determine the effectiveness of the therapeutic imagery administered to the patient, and patient imagery effectiveness data is generated representing the effectiveness of the therapeutic imagery for the patient. In one embodiment, the patient imagery effectiveness data and patient data associated with the patient are analyzed to generate one or more patient profiles. As used herein, the term “patient data” may include data associated with the patient, such as, but not limited to age, sex, ethnicity, marital status, income level, geographic location, personal and family medical history, including the current medical issue that the therapy is designed to treat. In one embodiment, patient data may also include patient preference data, which in some embodiments, may be tied to preferences for specific therapeutic imagery attributes. As used herein, the term “therapeutic imagery attributes” or “therapeutic imagery definition data” may include attributes that define the therapeutic imagery, such as the subject, composition, sentiments, and sensory perceptions associated with the therapeutic imagery. As used herein, the term “patient profile” may include models or templates that describe a particular type of patient.

In one embodiment, historical therapeutic imagery data is correlated with patient profile data to generate therapeutic imagery effectiveness model training data, which is used as training data to train one or more machine learning based models. In one embodiment, the machine learning based models are models that predict the effectiveness of given therapeutic imagery, and training the models with the therapeutic imagery effectiveness model training data results in the creation of one or more trained therapeutic imagery effectiveness prediction models. In various embodiments, the above described process may continue indefinitely, or may be terminated at any time at the discretion of an administrator of the method and system disclosed herein.

In various embodiments, once the therapeutic imagery effectiveness prediction models are trained, they can be used in a variety of ways. In one embodiment, the trained therapeutic imagery effectiveness prediction models can be used to dynamically generate improved or maximally effective therapeutic imagery for a specific patient, or a specific type of patient. In other embodiments, the trained therapeutic imagery effectiveness prediction models can be utilized independently of a specific patient, for example, to generate improved or maximally effective therapeutic imagery, which may be determined to be generally therapeutically effective for patients, regardless of the patient's background and history.

In one embodiment, in the case of dynamically generating improved or maximally effective therapeutic imagery for a specific patient, patient data associated with the patient and patient profile data associated with the predefined patient profiles are analyzed to select a patient profile that is the best match for the specific patient. In one embodiment, the selected patient profile data is provided to the trained therapeutic imagery effectiveness prediction models.

In one embodiment, new therapeutic imagery test data, representing new therapeutic imagery, is generated or otherwise obtained, wherein the new therapeutic imagery is new imagery to be considered for administration to one or more patients. In one embodiment, the new therapeutic imagery test data is provided to the trained therapeutic imagery effectiveness prediction models. In one embodiment, the trained therapeutic imagery effectiveness prediction models are utilized to generate predicted imagery effectiveness data for the new imagery represented by the new therapeutic imagery test data. In one embodiment, the predicted imagery effectiveness data associated with the new therapeutic imagery, and historical imagery effectiveness data associated with historical therapeutic imagery is analyzed to determine and select effective therapeutic imagery.

In one embodiment, once effective therapeutic imagery has been selected, effective imagery definition data associated with the effective therapeutic imagery is utilized to generate maximally effective therapeutic imagery for use in administration to a patient. In one embodiment, maximally effective imagery definition data associated with the maximally effective therapeutic imagery is incorporated into historical imagery definition data for future use in administration to a patient. This allows the maximally effective therapeutic imagery to be later used in the generation of model training data, so that the therapeutic imagery effectiveness prediction models can be continually trained with new data. In one embodiment, the maximally effective therapeutic imagery is then administered to the patient.

In one embodiment, in the case of generating improved or maximally effective therapeutic imagery that is generally effective for average patients, a process similar to that described above may be utilized without providing patient-specific profile data to the trained therapeutic imagery effectiveness prediction models. For example, in some embodiments, new therapeutic imagery test data is generated and provided to the trained therapeutic imagery effectiveness prediction models, and the trained therapeutic imagery effectiveness prediction models are utilized to generate predicted imagery effectiveness data for the new imagery. In some embodiments, the predicted imagery effectiveness data and the historical imagery effectiveness data are analyzed to select effective therapeutic imagery. Imagery definition data associated with the effective therapeutic imagery is then utilized to generate maximally effective therapeutic imagery. In one embodiment, maximally effective imagery definition data associated with the maximally effective therapeutic imagery is incorporated into historical imagery definition data for future use in administration to a patient and/or the maximally effective therapeutic imagery may also be administered to a patient.

The above described processes result in generation of maximally effective therapeutic imagery, which may then be administered to a patient, thus ensuring that the patient receives effective care, support, and treatment. Further, the above machine learning process employs a feedback loop, such that the therapeutic imagery effectiveness prediction models can be dynamically refined to account for newly received patient and effectiveness data, thus improving the accuracy of the effectiveness predictions generated by the models. In one embodiment the maximally effective therapeutic imagery can be generated in real-time, as it is being administered to the patient.

As a result of these and other disclosed features, which are discussed in more detail below, the disclosed embodiments provide an effective and efficient technical solution to the technical problem of dynamically generating therapeutic imagery to ensure that patients receive effective care, support, and treatment.

### BRIEF DESCRIPTION OF THE DRAWINGS

FIG. 1A is a block diagram of a training environment for creating trained therapeutic imagery effectiveness prediction models, in accordance with one embodiment.

FIG. 1B is a block diagram illustrating imagery data that may be utilized in order to generate maximally effective therapeutic imagery, in accordance with one embodiment.

FIG. 2A is a block diagram of a runtime environment for utilizing trained therapeutic imagery effectiveness prediction models to generate maximally effective therapeutic imagery for a specific patient, in accordance with one embodiment.

FIG. 2B is a simplified illustrative diagram of maximally effective therapeutic imagery being generated for a patient in real-time, in accordance with one embodiment.

FIG. 3 is a block diagram of a runtime environment for utilizing trained therapeutic imagery effectiveness prediction models to generate generalized maximally effective therapeutic imagery, in accordance with one embodiment.

FIG. 4 is a flow chart of a process for creating trained therapeutic imagery effectiveness prediction models, in accordance with one embodiment.

FIG. 5 is a flow chart of a process for utilizing trained therapeutic imagery effectiveness prediction models to generate maximally effective therapeutic imagery for a specific patient, in accordance with one embodiment.

FIG. 6 is a flow chart of a process for utilizing trained therapeutic imagery effectiveness prediction models to generate generalized maximally effective therapeutic imagery, in accordance with one embodiment.

Common reference numerals are used throughout the figures and the detailed description to indicate like elements. One skilled in the art will readily recognize that the above figures are merely illustrative examples and that other architectures, modes of operation, orders of operation, and elements/functions can be provided and implemented without departing from the characteristics and features of the invention, as set forth in the claims.

### DETAILED DESCRIPTION

Embodiments will now be discussed with reference to the accompanying figures, which depict one or more exemplary embodiments. Embodiments may be implemented in many different forms and should not be construed as limited to the embodiments set forth herein, shown in the figures, or described below. Rather, these exemplary embodiments are provided to allow a complete disclosure that conveys the principles of the invention, as set forth in the claims, to those of skill in the art.

### Term Definitions

As used herein, the term “patient” or “participant” may include an individual who has been diagnosed with one or more health conditions, an individual who is the recipient of a therapy and/or therapeutic imagery in a clinical or non-clinical setting, and/or an individual who has not been diagnosed with a health condition, but is a recipient of a therapy and/or therapeutic imagery in a clinical or non-clinical setting. Therefore, although the term “patient” will be used commonly throughout the enclosed specification, the term “participant” may also be used to indicate that applications of the methods and systems disclosed herein, used outside of a clinical setting, are also contemplated by the following disclosure.

As used herein the term “therapy,” “psychological therapy,” or therapeutic modality” may include psychological techniques, methodologies, and/or modalities utilized to treat a patient, such as, but not limited to psychotherapy, mind-body intervention, cognitive behavioral therapy (CBT), acceptance commitment therapy (ACT), dialectical behavioral therapy (DBT), exposure therapy, hypnotherapy, experiential therapy, and psychodynamic therapy.

As used herein, the term “mind-body intervention,” may include one or more therapeutic practices that employ a variety of techniques designed to facilitate the mind's capacity to affect bodily function and systems.

As used herein, the phrase “administration of a therapy,” “administering a therapy,” “administration of therapeutic imagery,” or “administering therapeutic imagery,” may include providing, and/or delivering, a therapy and/or therapeutic imagery to a patient. A therapy and/or therapeutic imagery may be administered to a patient directly by a health practitioner. A therapy and/or therapeutic imagery may be administered to a patient remotely, for example, over the internet or by computer software, without the direct involvement of a health practitioner. For example, the therapy and/or therapeutic imagery may be self-administered by the patient. A therapy and/or therapeutic imagery may also be administered to a patient remotely with partial involvement of a health practitioner. For example, the therapy and/or therapeutic imagery to be administered may be selected by a health practitioner, but the therapy and/or therapeutic imagery may then be self-administered by the patient, utilizing computer software, or the therapy and/or therapeutic imagery may be administered to the patient by computer software, but a health practitioner may monitor the patient's response data.

As used herein, the term “imagery,” “therapeutic imagery”, “guided imagery,” “guided meditation,” and “visualization may include scenes, scripts, or techniques that help a patient or participant generate mental images that simulate sensory perceptions and/or elicit strong emotions or feelings. Therapeutic imagery may include textual descriptions and/or visual images of places, objects, activities, colors, shapes, textures, sounds, smells and/or any other sensory experiences. Therapeutic imagery may be administered to a patient or participant via any type of audiovisual media, including but not limited to, spoken text, still images, video recordings, recordings of music, speech, and/or sound, and/or interactive media, such as through therapeutic software and virtual reality.

As used herein, the term “therapeutic imagery attributes” or “therapeutic imagery definition data” may include attributes that define the therapeutic imagery, such as the subject, composition, sentiments, and sensory perceptions associated with the therapeutic imagery, as will be discussed in further detail below.

As used herein, the terms “current therapeutic imagery” or “historical therapeutic imagery” may include imagery that has previously been generated, tested, established, and/or clinically validated for use in administration to a patient.

As used herein, the terms “new imagery,” and “new therapeutic imagery” may include imagery that has not been previously generated, tested, established, and/or clinically validated for use in administration to a patient. Additionally, the terms “new imagery,” and “new therapeutic imagery” may also include imagery that has been previously generated and/or tested, but may not yet be established and/or clinically validated for use in administration to a patient. In various embodiments, new therapeutic imagery may also include potential therapeutic imagery or candidate therapeutic imagery, in the sense that the imagery is being considered for administration to a patient.

As used herein, the terms “improved imagery” or “improved therapeutic imagery” may include new therapeutic imagery that has been found to be more effective than current or historical therapeutic imagery when used to treat one or more patients, wherein effectiveness of particular therapeutic imagery is determined by a variety of clinically validated outcome measures, as will be discussed in additional detail below.

As used herein, the terms “maximally effective imagery” or “maximally effective therapeutic imagery” may include therapeutic imagery that has been determined to be the most effective therapeutic imagery, for a particular period of time, out of the new, current, and/or historical therapeutic imagery of comparable type, wherein effectiveness of particular therapeutic imagery is determined by a variety of clinically validated outcome measures, as will be discussed in additional detail below. Improved therapeutic imagery and/or maximally effective therapeutic imagery may be the most effective, during the particular period of time, for the general patient population, the most effective for a particular group of patients, and/or the most effective for a specific individual patient, and the system and method disclosed herein accounts for these differences according to predefined guidelines, as will be discussed in further detail below.

As used herein, in various embodiments, the terms “patient response data” or “patient imagery response data” may include direct verbal or written feedback from the patient, indirect feedback, such as an indication of whether particular therapeutic imagery appears to be having an effect on the patient, and/or other measureable data such as, but not limited to, physiological sensor data and/or click-stream data showing patient engagement with the therapeutic imagery.

As used herein, the term “patient data” may include data associated with a patient, such as, but not limited to age, sex, ethnicity, marital status, income level, geographic location, personal and family medical history, including the current medical issue that the therapeutic imagery is designed to treat. In one embodiment, patient data may also include patient preference data, which in some embodiments, may be tied to preferences for specific therapeutic imagery attributes.

As used herein, the term “patient profile” may include models or templates that describe a particular type of patient.

### System

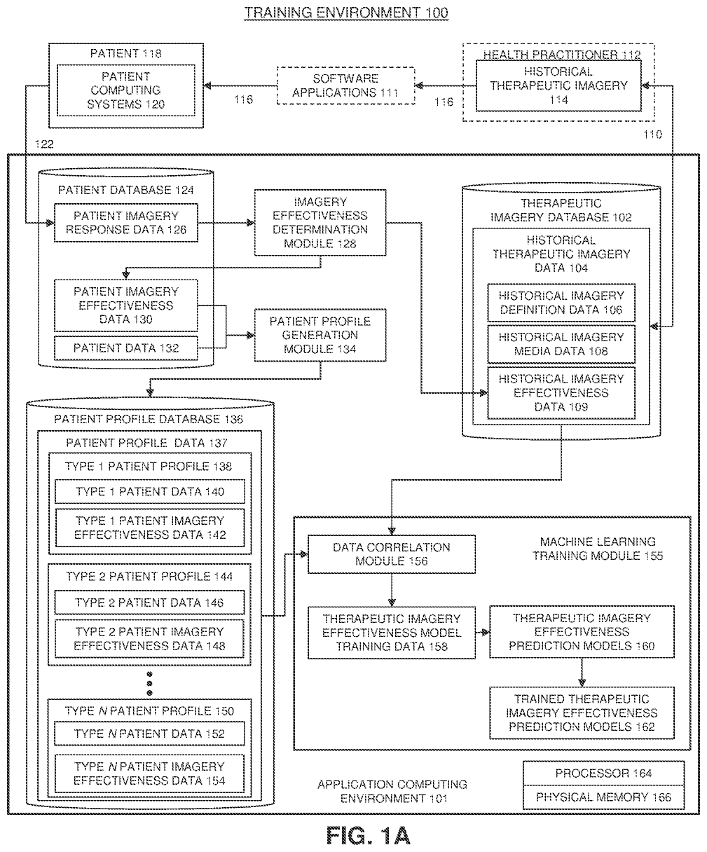

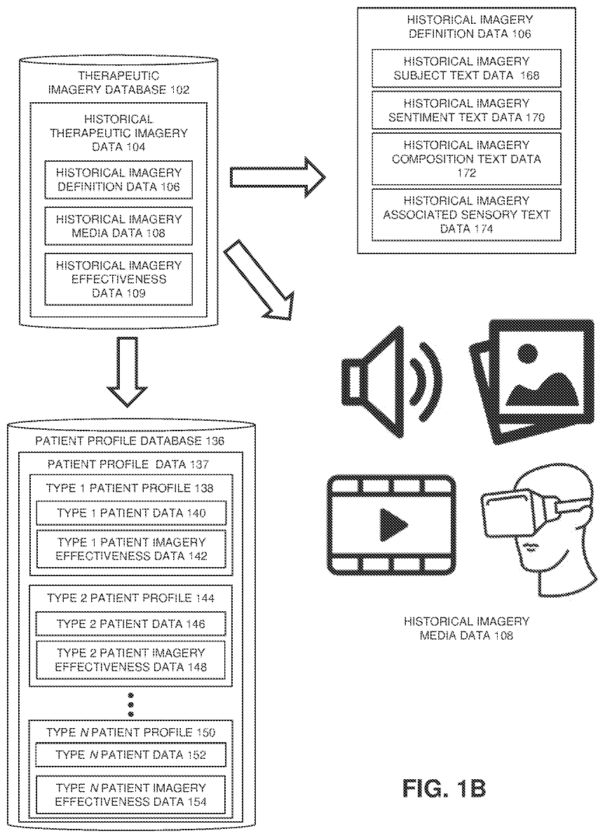

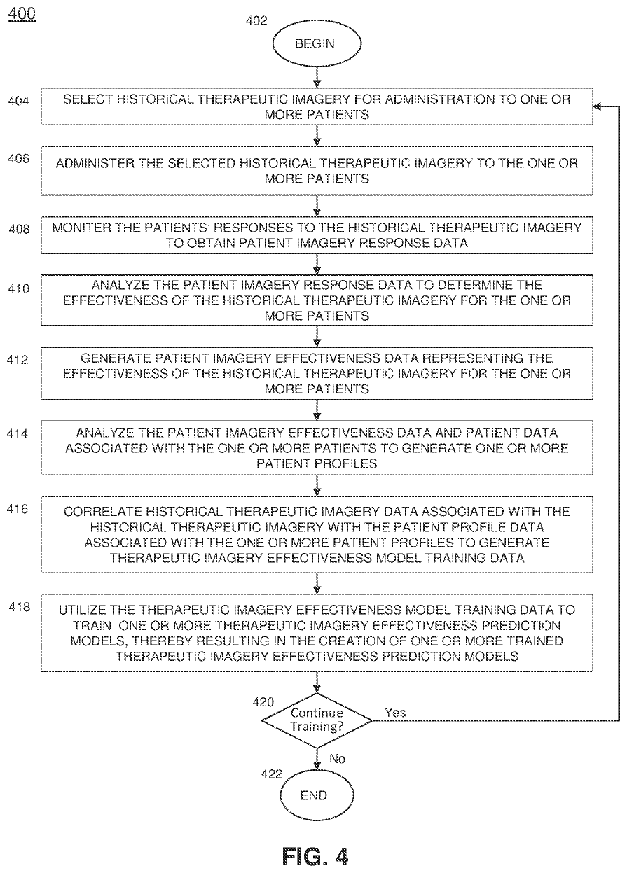

Embodiments of the present disclosure provide a technical solution to the technical problem of dynamically generating therapeutic imagery to ensure that patients receive effective care, support, and treatment. In the disclosed embodiments, historical therapeutic imagery is selected and administered to a patient, and the patient's responses to the historical therapeutic imagery are monitored to obtain patient imagery response data. In one embodiment the patient imagery response data is analyzed to determine the effectiveness of the therapeutic imagery, and patient imagery effectiveness data is generated representing the effectiveness of the therapeutic imagery. In one embodiment, the patient imagery effectiveness data and patient data associated with the patient are analyzed to generate one or more patient profiles. In one embodiment, historical therapeutic imagery data is correlated with patient profile data to generate therapeutic imagery effectiveness model training data, which is used as training data to train one or more machine learning based models, resulting in the creation of one or more trained therapeutic imagery effectiveness prediction models. The above described system and process will be discussed in additional detail below with reference to the system of FIG. 1A and the process of FIG. 4.

FIG. 1A is a block diagram of a training environment 100 for creating trained therapeutic imagery effectiveness prediction models, in accordance with one embodiment.

In various embodiments, training environment 100 includes application computing environment 101, historical therapeutic imagery 114, health practitioner 112, software applications 111, patient 118 and associated patient computing systems 120. In one embodiment, training environment 100 further includes communications channel 110, which facilitates retrieval of historical therapeutic imagery data from application computing environment 101, communications mechanisms 116, which facilitates administration of historical therapeutic imagery 114 to patient 118, and communications channel 122, which facilitates transmission of data from patient 118 to application computing environment 101. Each of the above listed elements will be discussed in further detail below.

In various embodiments, application computing environment 101 includes therapeutic imagery database 102, patient database 124, and patient profile database 136. In one embodiment, therapeutic imagery database 102 includes historical therapeutic imagery data 104, which further includes historical imagery definition data 106, historical imagery media data 108, and historical imagery effectiveness data 109. In one embodiment, patient database 124 includes patient imagery response data 126, patient imagery effectiveness data 130, and patient data 132. In one embodiment, patient profile database 136 includes patient profile data 137, which further includes type 1 patient profile 138, type 2 patient profile 144 through type n patient profile 150. In one embodiment, type 1 patient profile 138 further includes type 1 patient data 140 and type 1 patient imagery effectiveness data 142, type 2 patient profile 144 further includes type 2 patient data 146 and type 2 patient imagery effectiveness data 148, and type n patient profile 150 further includes type n patient data 152 and type n patient imagery effectiveness data 154. Each of the above listed elements will be discussed in further detail below.

In various embodiments, application computing environment 101 further includes several process modules, such as imagery effectiveness determination module 128, patient profile generation module 134, and machine learning training module 155. In one embodiment, machine learning training module 155 further includes data correlation module 156, therapeutic imagery effectiveness model training data 158, therapeutic imagery effectiveness prediction models 160, and trained therapeutic imagery effectiveness prediction models 162. In one embodiment, application computing environment 101 further includes processor 164 and physical memory 166, which together coordinate the operation and interaction of the data and data processing modules associated with application computing environment 101. Each of the above listed elements will be discussed in further detail below.

In one embodiment, patient 118 is a patient who has been diagnosed with a medical condition, and a determination is made as to whether patient 118 will benefit from treatment involving the use of therapeutic imagery. In one embodiment, the determination is made by health practitioner 112. In one embodiment, the determination is made by patient 118. In one embodiment, the determination is made by computer software algorithms. In various other embodiments, the determination may be made by one or more third parties. As one specific example, symptoms associated with a gastrointestinal (GI) disorder, such as irritable bowel syndrome (IBS), are often triggered by stress, and psychological issues such as, but not limited to, stress, anxiety, and depression, can worsen those symptoms. Patients who are suffering from stress, anxiety, and/or depression related to their medical diagnosis often benefit from receiving certain types of psychological therapies to help them better understand and manage their physiological and/or psychological conditions. Examples of therapeutic modalities that may be beneficial to patients include, but are not limited to, psychotherapy, mind-body intervention, cognitive behavioral therapy (CBT), acceptance commitment therapy (ACT), dialectical behavioral therapy (DBT), exposure therapy, hypnotherapy, experiential therapy, and psychodynamic therapy. One tool often utilized by health practitioners, typically as part of one or more therapy sessions, is that of therapeutic imagery, which may also be referred to as guided imagery, guided meditation, or visualization. Therapeutic imagery is often classified as a “mind-body intervention,” which refers to one or more practices that employ a variety of techniques designed to facilitate the mind's capacity to affect bodily function and systems. There are many documented scientific studies that show benefits of mind-body interventions in contributing to the treatment of a wide range of conditions including but not limited to, chronic pain, stress, anxiety, and depression.

Therapeutic imagery may be administered to a patient as a stand-alone treatment, or may be incorporated into a particular type of therapy, such as CBT. Even though a behavioral therapy, such as CBT, can be implemented in a variety of ways, it is typically administered utilizing a set of structured sessions or modules, which are each designed to teach a patient skills that enable the patient to better understand and manage thoughts and behaviors that may be negatively affecting their mental and physical states. Although aspects of CBT may be fairly structured, there is still enough flexibility to allow CBT to be adapted for use in treatment of particular conditions. One such example is that of utilizing CBT to treat IBS. The rationale for applying CBT to treat IBS is grounded in the biopsychosocial model, which states that one's biology, thoughts, emotions, external events, and behaviors influence IBS symptom expression in a bidirectional way.

Typically a trained practitioner uses therapeutic imagery to help a patient or participant generate mental images that simulate sensory perceptions and/or elicit strong emotions or feelings. Therapeutic imagery may encompass textual descriptions and/or visual images of places, objects, activities, colors, shapes, textures, sounds, smells and/or any other sensory experiences. Therapeutic imagery may be administered to a patient or participant via any type of audiovisual media, including but not limited to, spoken text, still images, video recordings, recordings of music, speech, and/or sound, and/or interactive media, such as through therapeutic software and virtual reality. Traditionally, in clinical settings, a health practitioner, such as health practitioner 112, may determine that a patient would benefit from the use of therapeutic imagery as part of a therapeutic treatment, and so health practitioner 112 may select one or more therapeutic imagery scenes, scripts, or techniques for administration to a patient. In some embodiments, especially in non-clinical settings, the therapeutic imagery scenes, scripts, or techniques may be selected by patient 118, by one or more software algorithms, and/or by one or more third parties.

The embodiments disclosed herein utilize machine learning models to generate improved and/or maximally effective therapeutic imagery for administration to one or more patients. In order to utilize machine learning models to generate improved and/or maximally effective therapeutic imagery, one or more therapeutic imagery effectiveness prediction models must first be trained, which is depicted in training environment 100 of FIG. 1A.

In one embodiment, once a determination has been made that patient 118 is likely to benefit from therapeutic imagery, historical therapeutic imagery 114 may be selected for administration to patient 118. As used herein, the term “historical therapeutic imagery” may include imagery that has previously been generated, tested, established, and/or clinically validated for use in administration to patients. In one embodiment, the determination as to which therapeutic imagery to administer to patient 118 may be made at the discretion of health practitioner 112, who, in some embodiments, is the health practitioner responsible for the treatment of patient 118, and the determination may be based on a wide variety of factors, such as, but not limited to, the severity of the patient's symptoms, the patient's medical history, the patient's known preferences, the patient's age group, the patient's sex, and the patient's ethnicity.

Once historical therapeutic imagery 114 has been selected for administration to patient 118, historical therapeutic imagery data 104 may be retrieved from therapeutic imagery database 102. Historical therapeutic imagery data 104 may include data such as historical imagery definition data 106, which defines any number of therapeutic imagery attributes. In the embodiments disclosed herein, it is expected that the number of therapeutic imagery attributes associated with historical imagery definition data 106 will be a very large number, due to the millions of ways of defining and combining imagery attributes, as noted above. In one embodiment, historical therapeutic imagery data 104 also includes historical imagery image data 108 and historical imagery effectiveness data 109, which will be discussed in additional detail below.

FIG. 1B is a block diagram illustrating therapeutic imagery data that may be utilized to generate maximally effective therapeutic imagery, in accordance with one embodiment.

Referring to FIG. 1A and FIG. 1B together, in one embodiment, historical therapeutic imagery data 104 may be utilized to train one or more therapeutic imagery effectiveness prediction models 160, which may then later be utilized to generate maximally effective therapeutic imagery.

As discussed above, in various embodiments, historical therapeutic imagery data 104 of therapeutic imagery database 102 may include historical imagery definition data 106, which defines one or more historical therapeutic imagery attributes associated with the historical therapeutic imagery. For example, in one embodiment, historical imagery definition data 106 may include historical imagery subject text data 168, which in various embodiments may include one or more textual descriptions of the subject of the imagery (e.g., “tropical beach scene on a sunny day with boats in the distance,” “redwood forest in the mountains on a foggy day,” “young woman jogging through a park,” “white kitten sleeping,” “galaxy fractals,” “abstract circles and spheres,” etc.) It should be noted that the above simplified examples are given for illustrative purposes only, and are not meant to limit the scope of the invention as disclosed herein, and as claimed below. One of skill in the art will readily appreciate that there are many ways to describe the subject of imagery, and many images contain multiple subjects, or no defined subject. In the case of video recordings, the imagery may contain hundreds or thousands of subjects, which are changing throughout the recording. Historical imagery subject text data 168 may include a list of a few key words and/or phrases, a list of hundreds of key words and/or phrases, or may include paragraphs or even pages of structured descriptive text.

In one embodiment, historical imagery definition data 106 may further include historical imagery sentiment text data 170, which in various embodiments may include one or more textual descriptions of the sentiment, mood, emotion, or feeling that the imagery is meant to convey (e.g. “relaxing,” “serene,” “empowering,” “joyful,” “engaged,” “curious”, etc.) Again, it should be noted that the above simplified examples are given for illustrative purposes only, and are not meant to limit the scope of the invention as disclosed herein, and as claimed below. One of skill in the art will readily appreciate that the sentiments conveyed by imagery are highly subjective, and many images convey multiple sentiments, which may be described or defined differently by different people. Historical imagery sentiment text data 170 may include a list of a few key words and/or phrases, a list of hundreds of key words and/or phrases, or may include paragraphs or even pages of structured descriptive text. Additionally, given the subjective nature of imagery, the historical imagery sentiment text data 170 may also include data indicating a percentage or number of people who classified particular imagery in a particular way. For instance, one person who enjoys the beach might describe a tropical beach scene as “relaxing,” while another person who doesn't enjoy the beach might describe the same tropical beach scene as “uncomfortable.” Likewise, one person who loves cats might describe imagery of a kitten sleeping as “joyful,” while a person who is allergic to cats, might describe the same imagery as “fearful.” Thus the historical imagery sentiment text data 170 may indicate, for example, that while 90% of people find beach scenes relaxing, the other 10% do not, and while 70% of people finds kittens to be joyful, the other 30% do not. In various embodiments, the historical imagery sentiment text data 170 may simply include positive, negative, or neutral sentiments, with ratings given on a scale from extremely negative to extremely positive. Thus, there are many different ways in which the historical imagery sentiment text data 170 may be defined and/or represented.

In one embodiment, historical imagery definition data 106 may further include historical imagery composition text data 172, which in various embodiments may include data describing the composition of imagery, such as, but not limited to, shapes, lines, curves, patterns, textures, colors, tone, contrast, lighting, viewpoint, depth, balance, and/or use of space. As noted above, historical imagery composition text data 172 may include a list of a few key words and/or phrases, a list of hundreds of key words and/or phrases, or may include paragraphs or even pages of structured descriptive text.

In one embodiment, historical imagery definition data 106 may further include historical imagery associated sensory text data 174, which in various embodiments may include textual descriptions of sensory data associated with the imagery. For example, if historical imagery subject text data 168 for particular historical imagery indicates “a tropical beach scene,” then historical imagery associated sensory text data 174 may include descriptions such as “the sound of waves and seagulls,” “the smell of sea air and sunscreen,” and/or “the feel of sand between toes and the warmth of sun on skin.” As noted above, the above examples are given for illustrative purposes only. In various embodiments, historical imagery associated sensory text data 174 may include a list of a few key words and/or phrases, a list of hundreds of key words and/or phrases, or may include paragraphs or even pages of structured descriptive text.

In various embodiment, historical therapeutic imagery data 104 of therapeutic imagery database 102 also includes historical imagery media data 108, which may include any number of digital media representations of the imagery described textually by historical imagery definition data 106. For example, for imagery related to a beach scene, historical imagery media data 108 may include audio recordings of waves crashing and seagulls, with vocal narration describing the scene, and/or may also include one or more digital or physical images of a coastline on a sunny day. For imagery related to a wooded forest scene, historical imagery media data 108 may include a video recording, in a first person view, of someone taking a peaceful walk on a trail through the woods, accompanied by the sounds of forest birds chirping. Alternatively, historical imagery media data 108 might be programmed into interactive computer software that allows a person using desktop applications, video games, or virtual reality devices to guide themselves through any number of settings, such as down a trail in a lush forest on a peaceful alien planet, where they may be able to interact with various elements of their surroundings.

It should be again noted that the above simplified examples are given for illustrative purposes only, and are not meant to limit the scope of the invention as disclosed herein, and as claimed below. One of skill in the art will readily appreciate that there are many ways to present and/or administer therapeutic imagery to a patient or a participant, including those described above, and/or any other type of therapeutic imagery delivery mechanism discussed herein, known at the time of filing, developed/made available after the time of filing, or any combination thereof.

In one embodiment, historical therapeutic imagery data 104 also includes historical imagery effectiveness data 109, which quantifies the effectiveness of the therapeutic imagery represented by historical imagery definition data 106 and historical imagery media data 108.

In one embodiment, historical imagery effectiveness data 109 includes generalized effectiveness data, while in other embodiments, historical imagery effectiveness data 109 may include patient-specific or profile-specific imagery effectiveness data, such as might be stored in patient profile database 136. For example, type 1 patient imagery effectiveness data 142 of type 1 patient profile 138 may indicate how effective historical therapeutic imagery data is for a patient that is classified as a type 1 patient, and patient imagery effectiveness data 142 may be represented as part of historical imagery effectiveness data 109. Patient profile database 136, and related patient imagery effectiveness data will be discussed in additional detail below.

Returning now to FIG. 1A, in various embodiments, once the historical therapeutic imagery data 104 is retrieved from therapeutic imagery database 102, historical therapeutic imagery 114 may be administered to patient 118 using one or more communication mechanisms 116. In some embodiments, communication mechanisms 116 include health practitioner 112 conducting a physical in-person meeting with patient 118 to verbally guide patient 118 through the historical therapeutic imagery 114. In other embodiments, communication mechanisms 116 include administering the historical therapeutic imagery 114 to patient 118 remotely, for example through a website, or through one or more software applications that can be executed from patient computing systems 120. In one embodiment, historical therapeutic imagery 114 may be administered to patient 118 directly by health practitioner 112. In one embodiment, historical therapeutic imagery 114 may be administered to patient 118 remotely, without the direct involvement of health practitioner 112. For example, historical therapeutic imagery 114 may be self-administered by patient 118. In one embodiment, historical therapeutic imagery 114 may also be administered to patient 118 remotely with partial involvement of health practitioner 112. For example, historical therapeutic imagery 114 may be selected for administration by health practitioner 112, but historical therapeutic imagery 114 may then be self-administered by patient 118, utilizing software applications 111, or historical therapeutic imagery 114 may be administered to patient 118 by software applications 111, but health practitioner 112 may monitor patient 118's response data.

In various embodiments, patient computing systems 120 may include, but are not limited to, a desktop computing system, a mobile computing system, a virtual reality computing system, a gaming computing system, a computing system that utilizes one or more Internet of Things (IoT) devices, and/or any other type of computing system discussed herein, known at the time of filing, developed/made available after the time of filing, or any combination thereof.

In one embodiment, once the selected historical therapeutic imagery 114 is administered to patient 118, patient 118's responses to the historical therapeutic imagery 114 are monitored to obtain patient imagery response data 126, which, in some embodiments, may then be stored in a data structure, such as patient database 124. As used herein, in various embodiments, “patient response data” or “patient imagery response data” may include feedback from the patient related to the historical therapeutic imagery 114 administered to patient 118. Patient imagery response data 126 may include direct verbal or written feedback, indirect feedback, such as an indication of whether particular therapeutic imagery appears to be having an effect on patient 118. The patient imagery response data 126 may further include any other measureable data such as, but not limited to, click-stream data showing details related to patient engagement with the therapeutic imagery, such as, but not limited to, the time that the patient spends engaging with the therapeutic imagery. The patient imagery response data 126 may also include data received from devices such as, but not limited to, sleep trackers, or other types of physiological sensors that may be used to measure the physiological state of patient 118, such as heart rate, respiratory rate, and/or blood pressure. The patient imagery response data 126 may further include data such as facial expressions, eye movement, and/or other measurable types of physical movement. The patient imagery response data 126 may then be provided to imagery effectiveness determination module 128, which in one embodiment is responsible for analyzing the patient imagery response data 126 to determine and assign effectiveness ratings to historical therapeutic imagery 114. Effectiveness ratings may be assigned to historical therapeutic imagery 114 as a whole, or may be assigned to various attributes of historical therapeutic imagery 114.

As one specific illustrative example, a patient who is suffering with chronic pain may be administered therapeutic imagery that is intended to shift the patient's focus away from the pain. A wide variety of data can be collected for classification as patient imagery response data 126. For example, health practitioner 112 may solicit direct feedback from patient 118 after completion of a therapy session in which historical therapeutic imagery 114 is administered, such as in a verbal interaction with patient 118, or through a survey or questionnaire administered in person or remotely, which asks patient 118 related questions, such as “Was there anything you particularly liked about the therapeutic imagery?” or “Was there anything you didn't like about the therapeutic imagery?” A patient may also provide this type of information without solicitation. These types of questions provide one way to quantify the effectiveness of therapeutic imagery. Indirect response data may also be collected. Continuing the above example, at some point after administration of the therapeutic imagery, the patient may be asked questions such as “Do you feel that your pain levels have increased, decreased, or stayed the same since your last session?” This type of question isn't specifically asking for the patient's direct feedback on the therapeutic imagery, but is instead designed to determine whether the therapeutic imagery has generated the intended result in the patient (e.g. decreased pain levels would be an indication that the imagery was effective). Additionally, data received from devices such as heart rate monitors, blood pressure monitors, and sleep trackers, may also provide indications as to whether the therapeutic imagery was effective for the patient. For example, if the therapeutic imagery is intended to decrease a patient's stress levels, physiological sensors can be used to determine a patient's stress levels during or after administration of the therapeutic imagery.

In practice, patient feedback, such as patient imagery response data 126, is typically collected in a structured manner using established clinical procedures to ensure the validity of the data interpretation. In various embodiments, the results of the data interpretation may sometimes be referred to as “clinically validated outcome measures,” which may typically be defined as tools that are used in clinical settings to assess the current status of a patient. With respect to the embodiments disclosed herein, analysis of clinically validated outcome measures for a patient can help to determine imagery effectiveness. In various embodiments, an effectiveness rating may be a measure of any number of factors, such as, but not limited to, whether particular imagery attributes or a combination of imagery attributes reduces symptom severity, eliminates symptoms, results in improved mood, and/or results in better quality of life for the patient.

In one embodiment, once imagery effectiveness determination module 128 has assigned one or more effectiveness ratings to historical therapeutic imagery 114, this data may also be stored in patient database 124 as patient imagery effectiveness data 130, for further use. In one embodiment, the patient imagery effectiveness data 130 generated by imagery effectiveness determination module 128 may also be stored in therapeutic imagery database 102 as historical imagery effectiveness data 109.

It should be noted again that the above examples are given for illustrative purposes, and are not intended to limit the scope of the invention as disclosed and claimed herein. One of ordinary skill in the art will readily appreciate that there are many different ways to determine and measure the effectiveness of therapeutic imagery. Administration of historical therapeutic imagery 114 discussed herein typically results in outcome measures that can be clinically validated and thus can reliably be associated with one or more related measures of effectiveness.

As to be expected however, although particular therapeutic imagery may be effective for the general patient population, or for the average patient, the same imagery may not be effective at all for a particular patient, or a particular type of patient. As one simplified example, imagery effectiveness determination module 128 might determine that therapeutic imagery depicting a tropical beach is 75% effective when administered to patient A, however therapeutic imagery depicting a wooded forest is determined to be 90% effective when administered to the same patient A. If the same therapeutic imagery is administered to patient B, it might be found that the tropical beach scene is 95% effective for patient B, whereas the wooded forest scene may only be 50% effective for patient B. Thus, it should be clear that the effectiveness ratings of historical therapeutic imagery 114 and its associated attributes are likely to vary significantly depending on the characteristics of the particular patient. Additionally, as discussed above, there are thousands of attributes that can be combined in millions of ways to form therapeutic imagery. As one simplistic example, one particular therapeutic imagery scene might depict a wooded forest with a gentle stream running through it, and the imagery might be administered to the patient verbally. Imagery effectiveness determination module 128 may determine that the wooded forest depiction was effective for that patient, but the vocal delivery was not effective for that patient, thus each attribute (e.g., in the above example, imagery attributes, and audio attributes) may be assigned separate effectiveness ratings.

It follows then, that in order to train one or more therapeutic imagery effectiveness prediction models, such as therapeutic imagery effectiveness prediction models 160, model training data that accounts for differences in imagery effectiveness among different types of patients must be gathered and assembled. In various embodiments, the system and method disclosed herein utilizes patient profile generation module 134 to build a plurality of patient profiles based on data such as patient data 132 of patient database 124. As used herein, the term “patient data” may include data associated with the patient, for example, patient characteristics such as, but not limited to age, sex, ethnicity, religion, marital status, income level, geographic location, personal and family medical history, including the current medical issue that the therapy is designed to treat. Patient data 132 may also include known visual and/or audio preferences of the patient.

In one embodiment, patient profile generation module 134 may correlate patient imagery effectiveness data 130 with specific profiles in the plurality of generated patient profiles, and, in one embodiment, may store the patient profile data in a data structure, such as patient profile database 136 for later use. Patient profile generation module 134 may generate any number of patient profiles, which may be characterized by the various combinations of patient characteristics represented by patient data 132. As shown in the illustrative embodiment of FIG. 1A, patient profile data 137 of patient profile database 136 includes type 1 patient profile 138, and type 2 patient profile 144 through type n patient profile 150. In one embodiment, type 1 patient profile 138 includes type 1 patient data 140 and type 1 patient imagery effectiveness data 142, type 2 patient profile 144 includes type 2 patient data 146 and type 2 patient imagery effectiveness data 148, and type n patient profile 150 includes type n patient data 152 and type n patient imagery effectiveness data 154, where n can represent any number of patient profiles, depending on the number of patient groupings that a user of the method and system disclosed herein wishes to create.

As specific illustrative examples, type 1 patient data 140 of type 1 patient profile 138 may describe a patient who is a male, between the ages of 10 and 15, living on the west coast of the United States, who has been diagnosed with irritable bowel syndrome (MS). Type 2 patient data 146 of type 2 patient profile 144 may describe a patient who is a female, between the ages of 55 and 60, living in London, who has been diagnosed with breast cancer. Type n patient data 152 of type n patient profile 150 may describe a patient who is a male, between the ages of 65 and 70, living in the southeastern part of the United States, who has been diagnosed with post-traumatic stress disorder (PTSD). In some embodiments, a patient profile of patient profile data 137 may describe a specific patient instead of a group of patients.

In various embodiments, the patient imagery effectiveness data, such as type 1 patient imagery effectiveness data 142, would represent a measure of how effective particular imagery is for patients of that particular type. In one embodiment, patient imagery effectiveness data may include a list of hundreds, thousands, or millions of imagery attributes and/or attribute combinations, each with corresponding data indicating an effectiveness rating for each imagery attribute or combination of attributes. In some embodiments, an effectiveness rating for an imagery attribute or combination of attributes among a particular type of patient may be a single number representing an average of the effectiveness ratings for that imagery attribute across all members of the group of patients defined by the patient profile type. In some embodiments an effectiveness rating for an imagery attribute among a particular type of patient may be a range of numbers representing the effectiveness ratings for that imagery attribute across all members of the group of patients defined by the patient profile type. In various other embodiments, a weighting system might be utilized, for instance to give higher weight to effectiveness ratings that are more common than others. For example, a particular imagery attribute might have a wide range of effectiveness values for a particular patient profile type, for example, from 30% to 70% effectiveness, however it may be the case that only one or two patients were associated with the 30% effectiveness rating and only one or two patients were associated with the 70% effectiveness rating, however most patients for that profile type were associated with a 60% rating, and so the 60% rating would receive a higher weight that than the other ratings. It should be noted again here that the above examples are given for illustrative purposes only and are not intended to limit the scope of the invention as disclosed and claimed herein.

In various embodiments, once a plurality of patient profiles are generated and associated with imagery effectiveness data, data correlation module 156 of machine learning training module 155 collects patient profile data 137 from patient profile database 136, and historical therapeutic imagery data 104 from therapeutic imagery database 102 and correlates the data to prepare it for transformation into therapeutic imagery effectiveness model training data 158.

In various embodiments, and largely depending on the machine learning based models used, the patient profile data 137 and/or the historical therapeutic imagery data 104 is processed using various methods know in the machine learning arts to identify elements and to vectorize the patient profile data 137 and/or the historical therapeutic imagery data 104. As a specific illustrative example, in a case where the machine leaning based model is a supervised model, the historical therapeutic imagery data 104 and the patient profile data 137 can be analyzed and processed to identify individual elements found to be indicative of imagery effectiveness among certain types of patients, or among a generalized population of patients. These individual elements are then used to create imagery effectiveness data vectors in multidimensional space, resulting in therapeutic imagery effectiveness model training data 158. Therapeutic imagery effectiveness model training data 158 is then used as input data for training one or more machine learning models, such as therapeutic imagery effectiveness prediction models 160. The imagery effectiveness data for a patient profile that correlates with the imagery effectiveness data vector associated with that patient profile is then used as a label for the resulting vector. In various embodiments, this process is repeated for each imagery attribute defined by historical imagery definition data 106 of therapeutic imagery database 102, and for each patient profile type represented by patient profile data 137 of patient profile database 136. The result is that multiple, often millions, of correlated pairs of imagery effectiveness data vectors and patient profiles (as represented by therapeutic imagery effectiveness model training data 158) are used to train one or more machine learning based models, such as therapeutic imagery effectiveness prediction models 160. Consequently, this process results in the creation of one or more trained therapeutic imagery effectiveness prediction models 162.

Those of skill in the art will readily recognize that there are many different types of machine learning based models known in the art, and as such, it should be noted that the specific illustrative example of a supervised machine learning based model discussed above should not be construed as limiting the embodiments set forth herein.

For instance, in various embodiments, the one or more machine learning based models can be one or more of: supervised machine learning-based models; semi supervised machine learning-based models; unsupervised machine learning-based models; classification machine learning-based models; logistical regression machine learning-based models; neural network machine learning-based models; deep learning machine learning-based models; and/or any other machine learning based models discussed herein, known at the time of filing, or as developed/made available after the time of filing.

As will be discussed in further detail below, in various embodiments, once the therapeutic imagery effectiveness prediction models 160 are trained, they can be used in a variety of ways. In one embodiment, the trained therapeutic imagery effectiveness prediction models 162 can be used to dynamically generate improved or maximally effective therapeutic imagery for a specific patient, or for a specific type of patient. In one embodiment, in the case of dynamically generating improved or maximally effective therapeutic imagery for a specific patient, patient data associated with a patient and patient profile data associated with predefined patient profiles are analyzed to select a patient profile that is the best match for the specific patient, and the selected patient profile data is provided to the trained therapeutic imagery effectiveness prediction models 162. In one embodiment, new therapeutic imagery test data is generated or otherwise obtained, and is also provided to the trained therapeutic imagery effectiveness prediction models 162.

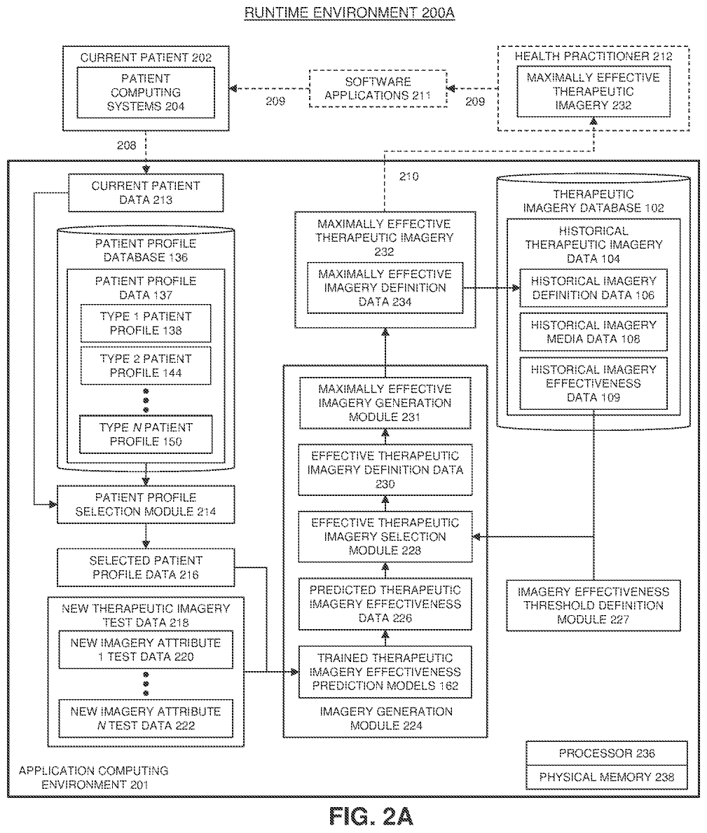

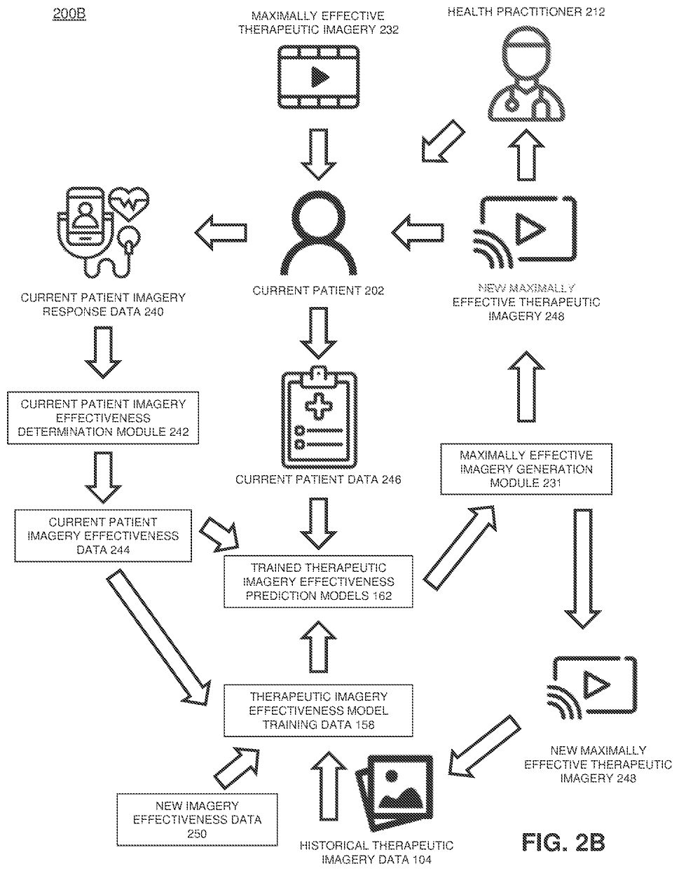

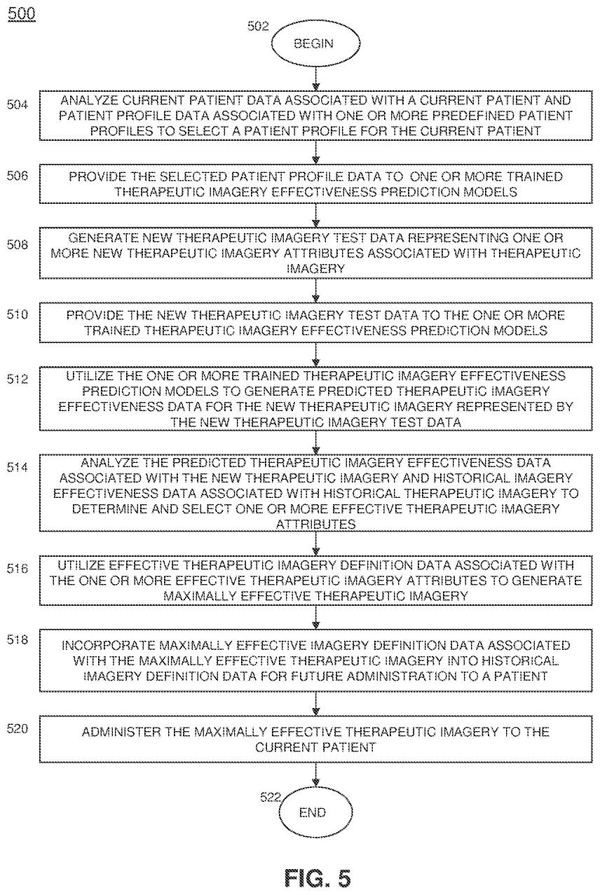

In one embodiment, the trained therapeutic imagery effectiveness prediction models 162 are utilized to generate predicted therapeutic imagery effectiveness data for the new imagery. In one embodiment, predicted imagery effectiveness data, and historical imagery effectiveness data are analyzed to determine and select effective therapeutic imagery attributes, which are utilized to generate maximally effective therapeutic imagery. In one embodiment, maximally effective imagery definition data associated with the maximally effective therapeutic imagery is incorporated into historical imagery definition data for future administration to patient. In one embodiment, the maximally effective therapeutic imagery is administered to the patient. The above described system and process will be discussed in additional detail below with reference to the system of FIG. 2A and the process of FIG. 5.

FIG. 2A is a block diagram of a runtime environment 200A for utilizing trained therapeutic imagery effectiveness prediction models to generate maximally effective therapeutic imagery for a specific patient, in accordance with one embodiment.

In various embodiments, runtime environment 200A includes application computing environment 201, current patient 202 and associated patient computing systems 204, software applications 211, health practitioner 212, and maximally effective therapeutic imagery 232. In one embodiment, maximally effective therapeutic imagery 232 includes maximally effective imagery definition data 234. In one embodiment, runtime environment 200A further includes communications channel 208, which facilitates transmission of data from current patient 202 to application computing environment 201, communications mechanisms 209, which facilitates administration of maximally effective therapeutic imagery 232 to current patient 202, and communications channel 210, which facilitates retrieval of data from application computing environment 201. Each of the above listed elements will be discussed in further detail below.

In various embodiments, application computing environment 201 includes therapeutic imagery database 102, and patient profile database 136. In one embodiment, therapeutic imagery database 102 includes historical therapeutic imagery data 104, which further includes historical imagery definition data 106, historical imagery media data 108, and historical imagery effectiveness data 109. In one embodiment, patient profile database 136 includes patient profile data 137, which further includes type 1 patient profile 138, and type 2 patient profile 144 through type n patient profile 150. Each of the above listed elements will be discussed in further detail below.

In various embodiments, application computing environment 201 further includes additional data such as current patient data 213, selected patient profile data 216, and new therapeutic imagery test data 218, which further includes new imagery attribute 1 test data 220 through new imagery attribute n test data 222. In one embodiment, application computing environment 201 further includes several process modules, such as patient profile selection module 214, imagery generation module 224, and imagery effectiveness threshold definition module 227. In various embodiments, imagery generation module 224 includes trained therapeutic imagery effectiveness prediction models 162, predicted therapeutic imagery effectiveness data 226, effective therapeutic imagery selection module 228, effective therapeutic imagery definition data 230, and maximally effective imagery generation module 231. In one embodiment, application computing environment 201 further includes processor 236 and physical memory 238, which together coordinate the operation and interaction of the data and data processing modules associated with application computing environment 201. Each of the above listed elements will be discussed in further detail below.

In one embodiment, current patient 202 is a patient who has been diagnosed with a medical condition, and a determination is made as to whether current patient 202 will benefit from receiving therapeutic imagery. In one embodiment, once a determination has been made that current patient 202 is likely to benefit from therapeutic imagery, the previously trained therapeutic imagery effectiveness prediction models 162 are utilized in order to generate therapeutic imagery that will be maximally effective for current patient 202.

In order to achieve this outcome, in one embodiment, current patient data 213 is obtained, either directly from current patient 202 and/or patient computing systems 204, from medical files associated with current patient 202 and/or current patient data 213 may be retrieved from a database of previously collected patient data. As noted above, and as used herein, the term “patient data” may include data associated with the patient, for example, patient characteristics such as, but not limited to age, sex, ethnicity, religion, marital status, income level, geographic location, personal and family medical history, including the current medical issue that the therapeutic imagery is designed to treat. In one embodiment, patient data may also include patient preference data, which in some embodiments, may be tied to preferences for specific therapeutic imagery attributes. In various embodiments, patient computing systems 204 may include, but are not limited to, a desktop computing system, a mobile computing system, a virtual reality computing system, a gaming computing system, a computing system that utilizes one or more Internet of Things (IoT) devices, or any combination thereof.

In some embodiments, there may be no specific current patient 202, and test data may be used in place of current patient data 213. For example, a theoretical patient may be contemplated, and data describing characteristics of the theoretical patient may be used as test data in place of current patient data 213 to generate maximally effective therapeutic imagery for the theoretical patient. In various embodiments, test data may be generated by one or more machine learning models that have been trained to predict effectiveness of the test data.

In various embodiments, once the current patient data 213 has been obtained for current patient 202, patient profile selection module 214 analyzes the current patient data 213 along with the patient profile data 137 of patient profile database 136, in order to select a patient profile that most closely matches the characteristics of current patient 202.

In one embodiment, patient profile selection module 214 compares various characteristics of current patient 202 to patient characteristics represented by the one or more patient profiles in the patient profile database 136. As will be noted by those of skill in the art, various mechanisms and algorithms may be utilized to determine similarities between current patient 202 and the patient profiles represented by patient profile data 137. Similarity of current patient 202 to a particular patient profile may be determined by any number of factors, such as, but not limited to current patient 202's age, sex, ethnicity, religion, marital status, income level, geographic location, personal and family medical history, including the current medical issue that the therapeutic imagery is designed to treat. In one embodiment, patient similarity may also be determined by patient preference data.

In one embodiment, one or more thresholds may be defined to determine how close of a match current patient 202 is to a particular patient profile. For example, if current patient 202's characteristics match with type 1 patient profile 138 by 60%, but match with type 2 patient profile 144 by 80%, and no other patient profile type is found to provide a better match, the patient profile with the closest match may be selected for utilization in determination of effective therapeutic imagery to utilize for treating current patient 202.

Continuing the specific illustrative examples given above, current patient 202 may be a male, age 13, living in California, who has been diagnosed with IBS, and so may be classified as a ‘type 1’ patient, wherein the ‘type 1’ patient is associated with type 1 patient profile 138, which describes a patient who is a male, between the ages of 10 and 15, living on the west coast of the United States, who has been diagnosed with IBS, and so patient profile selection module 214 may determine that current patient 202 should be associated with type 1 patient profile 138, and this selection may be represented by selected patient profile data 216. It should be noted herein that the above examples are simplified, and are given for illustrative purposes only. One of skill in the art will readily recognize that the millions of different combinations of patient characteristics, the models that govern the interactions between those characteristics and the millions of therapeutic imagery attributes and combinations of imagery attributes that make up the therapeutic imagery to be administered to those patients, requires a vast amount of data collection and analysis which simply cannot be performed by the human mind alone, even with the aid of pen and paper and even given unlimited time to accomplish the task.

In various embodiments, once patient profile selection module 214 has determined selected patient profile data 216, the selected patient profile data 216 is provided as input to the one or more trained therapeutic imagery effectiveness prediction models 162, along with new therapeutic imagery test data 218. As discussed above, in various embodiments, and as used herein, the terms “current therapeutic imagery” or “historical therapeutic imagery” may include therapeutic imagery that has previously been generated, tested, established, and/or clinically validated for use in administration to a patient. Likewise, in one embodiment, the terms “new imagery,” and “new therapeutic imagery” may include therapeutic imagery that has not been previously generated, tested, established, and/or clinically validated for use in administration to a patient. Additionally, in some embodiments, the terms “new imagery,” and “new therapeutic imagery,” and may refer to therapeutic imagery that has been previously generated and/or tested, but may not yet be established and/or clinically validated for use in administration to a patient. In various embodiments, new therapeutic imagery may also include potential therapeutic imagery or candidate therapeutic imagery, in the sense that it is imagery that is being considered for use in a therapy.

As one simplified example, historical therapeutic imagery used to treat patients suffering from anxiety may include depictions of nature scenes, delivered either visually or verbally. New therapeutic imagery used to treat patients suffering from anxiety may instead include abstract visualizations focusing on shape and color, delivered both visually and verbally. Similarly, historical therapeutic imagery used to treat patients may include visualizations delivered verbally, whereas new therapeutic imagery may include visual and audio content delivered in a virtual reality simulation.

In one embodiment, new therapeutic imagery test data 218 of FIG. 2A includes data representing any number of new therapeutic imagery attributes, which may be represented by new imagery attribute 1 test data 220 through new imagery attribute n test data 222. In one embodiment, once the selected patient profile data 216 and new therapeutic imagery test data 218 have been provided as input to the one or more trained therapeutic imagery effectiveness prediction models 162 of imagery generation module 224, the one or more trained therapeutic imagery effectiveness prediction models 162 generate predicted therapeutic imagery effectiveness data 226. In various embodiments, predicted therapeutic imagery effectiveness data 226 represents the predicted effectiveness of each of the new therapeutic imagery attributes represented by new therapeutic imagery test data 218 for a patient who matches the patient profile type represented by selected patient profile data 216.

As one simplified example, patient profile selection module 214 might categorize current patient 202 as a match for a type 1 patient, as represented by type 1 patient profile 138 of patient profile data 137. Predicted therapeutic imagery effectiveness data 226 might indicate that a first new imagery attribute is 75% effective for type 1 patients, and a second new imagery attribute is 50% effective for type 1 patients. Likewise, for a patient who has been categorized as a type 2 patient, predicted therapeutic imagery effectiveness data 226 might indicate that the same first new imagery attribute is 30% effective for type 2 patients, and the same second new imagery attribute is 90% effective for type 2 patients. Thus, the output of the trained therapeutic imagery effectiveness prediction models 162, predicted therapeutic imagery effectiveness data 226, is dependent on both the new therapeutic imagery test data 218, as well as the patient profile type represented by selected patient profile data 216. As discussed above, effectiveness of imagery attributes or combinations of imagery attributes can be determined and defined in a number of ways, including, but not limited to, analysis of direct or indirect feedback from a patient, analysis of patient physiological data, and/or analysis of a variety of clinically validated outcome measures. An effectiveness rating may be a measure of any number of factors, such as, but not limited to, whether an imagery attribute or a combination of imagery attributes reduces symptom severity, eliminates symptoms, results in improved mood, and/or results in better quality of life for the patient.

In one embodiment, once predicted therapeutic imagery effectiveness data 226 has been generated by trained therapeutic imagery effectiveness prediction models 162, it is passed to effective therapeutic imagery selection module 228 of imagery generation module 224 for further analysis. In one embodiment, effective therapeutic imagery selection module 228 selects one or more of the new therapeutic imagery attributes represented by new therapeutic imagery test data 218 that have been found to be effective. A determination as to what constitutes an “effective” imagery attribute may be made in any number of ways. As one illustrative example, imagery effectiveness threshold definition module 227 may set one or more threshold values for the effectiveness ratings represented by the predicted therapeutic imagery effectiveness data 226. In one embodiment, imagery effectiveness threshold definition module 227 may be separate from imagery generation module 224. In one embodiment, imagery effectiveness threshold definition module 227 may be a sub-module of imagery generation module 224. In one embodiment, one or more threshold values may be explicitly set, for example, based on input from one or more health practitioners. In various other embodiments, imagery effectiveness threshold definition module 227 may derive or learn one or more threshold values based on analysis of training data, such as, but not limited to historical imagery effectiveness data 109. As one simplified example, in one embodiment, imagery effectiveness threshold definition module 227 may define an effectiveness threshold such that any imagery attribute having a known or predicted effectiveness rating of 75% or higher should be considered an “effective” imagery attribute by effective therapeutic imagery selection module 228. In one embodiment, effective therapeutic imagery selection module 228 may also consider historical imagery effectiveness data 109 in determining and selecting effective imagery.

Continuing the above simplified example, historical therapeutic imagery used to treat patients suffering from anxiety may include depictions of nature scenes, delivered either visually or verbally. New therapeutic imagery used to treat patients suffering from anxiety may instead include abstract visualizations focusing on shape and color, delivered both visually and verbally. It may be found that the historical therapeutic imagery has a known effectiveness rating of 90%, whereas the new therapeutic imagery has a predicted effectiveness rating of 80%. In the illustrative embodiment where imagery effectiveness threshold definition module 227 has set the threshold value for effectiveness ratings to 75%, the effective therapeutic imagery selection module 228 may determine that, while the new imagery is predicted to be effective, the historical imagery is actually known to be more effective, and so the historical imagery may be selected over the new imagery. Similarly, historical therapeutic imagery used to treat patients may include visualizations delivered verbally, whereas new therapeutic imagery may include visual and audio content delivered in a virtual reality simulation. The historical therapeutic imagery may have a known 75% effectiveness rating, whereas the new therapeutic imagery may have a predicted 85% effectiveness rating, and so effective therapeutic imagery selection module 228 may select the new therapeutic imagery.

In one embodiment, once effective therapeutic imagery selection module 228 has selected one or more effective therapeutic imagery attributes, effective therapeutic imagery definition data 230 is generated, which contains data defining the one or more selected effective therapeutic imagery attributes. In one embodiment, maximally effective imagery generation module 231 utilizes effective therapeutic imagery definition data 230 to generate maximally effective therapeutic imagery 232. As used herein, the term “maximally effective imagery” or “maximally effective therapeutic imagery” may include therapeutic imagery that has been determined to be the most effective therapeutic imagery, for a particular period of time, out of the new, current, and/or historical effective therapeutic imagery. In various embodiments, maximally effective therapeutic imagery 232 may include any number and combination of effective therapeutic imagery attributes, and each of these therapeutic imagery attributes or attribute combinations is defined by maximally effective imagery definition data 234. Continuing the above illustrative example, imagery generation module 224 may determine that new therapeutic imagery including abstract shapes is maximally effective for the patient profile type represented by selected patient profile data 216, independently of other imagery attributes. Imagery generation module 224 may instead determine that new therapeutic imagery including abstract shapes is only effective for the patient profile type represented by selected patient profile data 216 when the shapes feature curved lines and are depicted as being different shades of blue.

It should be noted here that the above simplified examples are given for illustrative purposes only and are not intended to limit the invention as disclosed and claimed herein. It should be readily apparent to those of ordinary skill in the art that there are millions of potential imagery attributes and attribute combinations that may be utilized in therapeutic imagery, and so the generation of maximally effective imagery that takes into account the effectiveness of particular imagery attributes for particular patient characteristics is not a task that can be accomplished in the human mind, even with pen and paper, and even given unlimited time.

Referring briefly to FIG. 1A and FIG. 2A together, in various embodiments, once maximally effective therapeutic imagery 232 has been generated by maximally effective imagery generation module 231 of imagery generation module 224, the maximally effective imagery definition data 234 representing the maximally effective therapeutic imagery 232 may be stored in a data structure, such as therapeutic imagery database 102, for further use. For example, in one embodiment, maximally effective imagery definition data 234 is incorporated into historical imagery definition data 106 of historical therapeutic imagery data 104, which, in some embodiments may be stored in therapeutic imagery database 102. This is advantageous because it creates a feedback loop for the machine learning process, wherein the newly generated maximally effective therapeutic imagery 232 can be incorporated into the therapeutic imagery effectiveness model training data 158, of FIG. 1A, which is used to train the therapeutic imagery effectiveness prediction models 160. In this manner, the trained therapeutic imagery effectiveness prediction models 162 may be continually updated and refined as new patient imagery response data 126 is received from patient 118.

In one embodiment, once maximally effective therapeutic imagery 232 has been generated by maximally effective imagery generation module 231 of imagery generation module 224, the maximally effective therapeutic imagery 232 may then be administered to a patient, such as current patient 202. In some embodiments, once generated, the maximally effective therapeutic imagery 232 may be administered directly to current patient 202. In some embodiments, health practitioner 212 may review maximally effective therapeutic imagery 232 prior to administration to current patient 202. In some embodiments, the maximally effective therapeutic imagery 232 may be stored in a data structure, such as therapeutic imagery database 102, for further use, but might not be administered to current patient 202 and/or the maximally effective therapeutic imagery 232 may be administered to a patient other than current patient 202. In one embodiment, the maximally effective therapeutic imagery 232 may be administered to current patient 202 (or a patient other than current patient 202), and patient imagery response data may be collected in real-time such that new maximally effective therapeutic imagery may be generated and administered to current patient 202 in real-time, in reaction to changes in the current patient 202's response data, mental status, physical status, and/or other changes in associated current patient data, as will be described in additional detail below.

In various embodiments, the maximally effective therapeutic imagery 232 may be administered to current patient 202 using one or more communication mechanisms 209. In some embodiments, communication mechanisms 209 include health practitioner 212 conducting a physical in-person meeting with current patient 202 to verbally guide current patient 202 through the maximally effective therapeutic imagery 232. In other embodiments, communication mechanisms 209 include administering the maximally effective therapeutic imagery 232 to current patient 202 remotely, for example through a website, or through one or more software applications 211 that can be executed from patient computing systems 204. In one embodiment, maximally effective therapeutic imagery 232 may be administered to current patient 202 directly by health practitioner 212. In one embodiment, maximally effective therapeutic imagery 232 may be administered to current patient 202 remotely, without the direct involvement of health practitioner 212. For example, maximally effective therapeutic imagery 232 may be self-administered by current patient 202. In one embodiment, maximally effective therapeutic imagery 232 may also be administered to current patient 202 remotely with partial involvement of health practitioner 212.

In various embodiments, patient computing systems 204 may include, but are not limited to, a desktop computing system, a mobile computing system, a virtual reality computing system, a gaming computing system, a computing system that utilizes one or more Internet of Things (IoT) devices, or any combination thereof.

FIG. 2B is a simplified illustrative diagram 200B of maximally effective therapeutic imagery being generated for a patient in real-time, in accordance with one embodiment.

As depicted in the illustrative embodiment of FIG. 2B, in one embodiment, generation of new maximally effective therapeutic imagery may be performed in real-time such that the therapeutic imagery is reactive to various events, such as, but not limited to, changes in a patient's response data, mental status, physical status, and/or other changes associated with the patient or patient data.

Referring to FIGS. 1A, 2A, and 2B together, in one embodiment, maximally effective therapeutic imagery 232 (generated in FIG. 2A), is administered to current patient 202 (or a patient other than current patient 202), and current patient imagery response data 240 may be collected in real-time from current patient 202. As discussed above, in various embodiments, current patient imagery response data 240 may include, but is not limited to, direct verbal or written feedback from the patient, indirect feedback, such as an indication of whether particular therapeutic imagery appears to be having an effect on the patient, and/or other measureable data such as, but not limited to, physiological sensor data and/or click-stream data showing patient engagement with the therapeutic imagery.

In one embodiment, once current patient imagery response data 240 is collected, it is passed to current patient imagery effectiveness determination module 242, which functions similarly to imagery effectiveness determination module 128 of FIG. 1A, discussed above. Current patient imagery effectiveness determination module 242 may then generate current patient imagery effectiveness data 244, which, in the embodiment of FIG. 2B, represents the effectiveness for current patient 202 of one or more imagery attributes of maximally effective therapeutic imagery 232. In one embodiment, current patient data 213 from FIG. 2A, may be updated at any time during administration of maximally effective therapeutic imagery 232, to reflect any changes in the patient data associated with current patient 202. This is represented in FIG. 2B as current patient data 246.

In one embodiment, current patient imagery effectiveness data 244, and current patient data 246 are provided as inputs to trained therapeutic imagery effectiveness prediction models 162, along with any new imagery test data (not shown) and the resulting outputs are utilized by maximally effective imagery generation module 231 to generate new maximally effective therapeutic imagery 248. For example, current patient 202 may have initially been categorized as a type 2 patient, and maximally effective therapeutic imagery 232 may be generated based on type 2 patient data. Upon receipt of current patient imagery response data 240, or updated current patient data 246, current patient 202 may be reclassified as a different type of patient for generation of new maximally effective therapeutic imagery 248, and/or changes may be made to one or more attributes associated with maximally effective therapeutic imagery 232 to react to real-time changes in current patient imagery response data 240. In some embodiments, the entirety of maximally effective therapeutic imagery 232 may be changed and replaced with new maximally effective therapeutic imagery 248 (e.g. a shift in the subject of the imagery), while in other embodiments, only some of the imagery attributes of maximally effective therapeutic imagery 232 may be changed to transform maximally effective therapeutic imagery 232 into new maximally effective therapeutic imagery 248 (e.g. a shift in the color scheme of the imagery).

In one embodiment, trained therapeutic imagery effectiveness prediction models 162 are being continuously trained in real-time to account for newly received patient and imagery effectiveness data. For example, in one embodiment, patient and imagery data, such as current patient imagery effectiveness data 244, is incorporated into therapeutic imagery effectiveness model training data 158. In one embodiment, new maximally effective therapeutic imagery 248 is incorporated into historical therapeutic imagery data 104, which may then be administered to one or more patients to generate new imagery effectiveness data 250. New imagery effectiveness data 250 and historical therapeutic imagery data 104 may then also be incorporated into therapeutic imagery effectiveness model training data 158, which, in one embodiment, is used to provide additional training to trained therapeutic imagery effectiveness prediction models 162.

In some embodiments, new maximally effective therapeutic imagery 248 may be administered in real-time, directly to current patient 202. In other embodiments, new maximally effective therapeutic imagery 248 may first be directed to health practitioner 212 for review and/or validation. In one embodiment, health practitioner 212 may make a decision as to whether to administer the new maximally effective therapeutic imagery 248 to the current patient 202 and/or whether to take one or more other actions, such as, but not limited to, providing different therapeutic imagery to current patient 202, discarding new maximally effective therapeutic imagery 248, or incorporating new maximally effective therapeutic imagery 248 into historical therapeutic imagery data 104 for future use in administration to patients. It should be noted that the above simplified examples are given for illustrative purposes only are not intended to limit the scope of the invention as disclosed and as claimed herein.

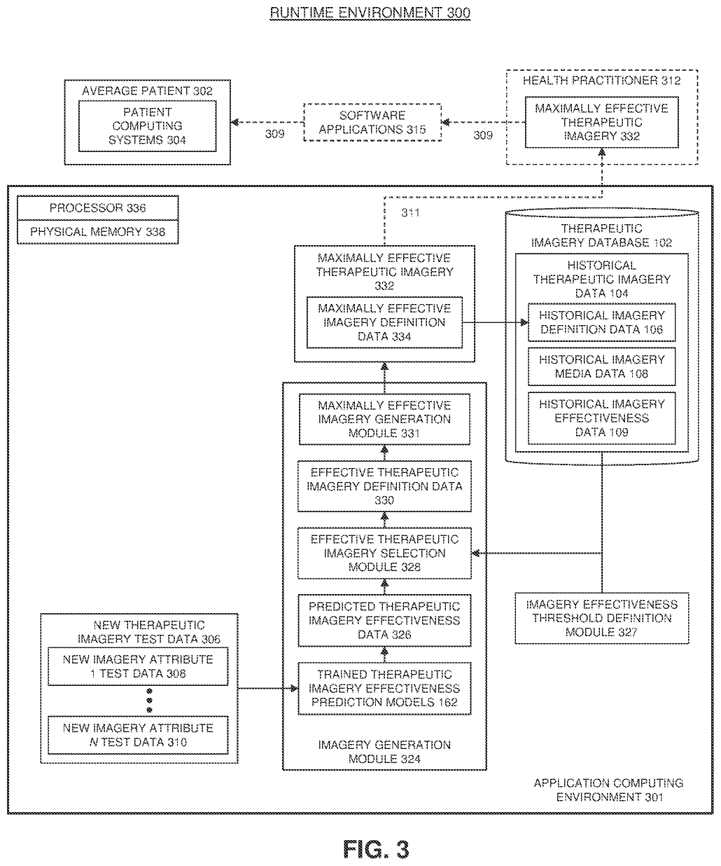

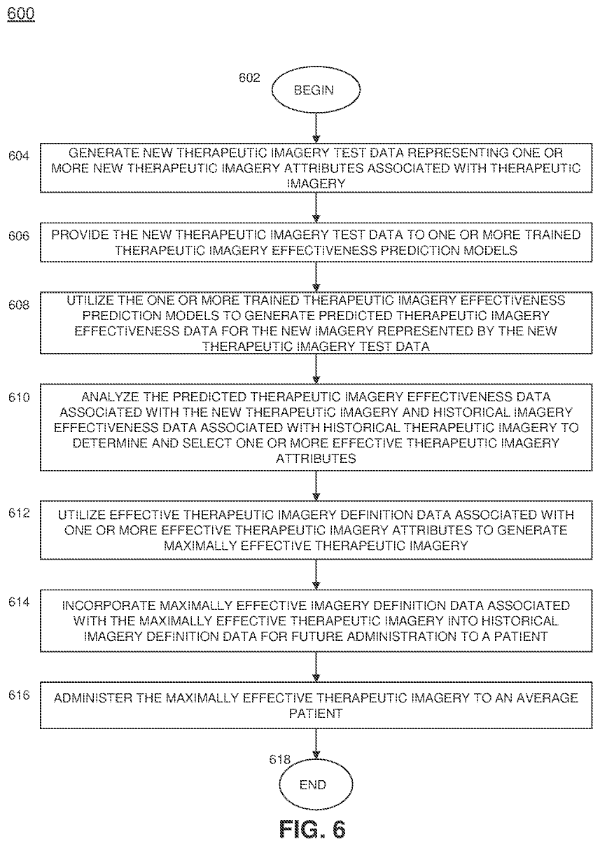

As will be discussed in further detail below, in addition to the embodiments discussed above, trained therapeutic imagery effectiveness prediction models 162 can be utilized independently of a specific patient or specific type of patient, for example, to generate improved or maximally effective therapeutic imagery that is generally effective for patients, regardless of the patients' background and history. In one embodiment, in the case of generating improved or maximally effective therapeutic imagery that is generally effective for patients, or is effective for an average patient, a system similar to that described above may be utilized, without providing patient-specific profile data to the trained therapeutic imagery effectiveness prediction models. For example, in some embodiments, new therapeutic imagery test data is generated and provided to the trained therapeutic imagery effectiveness prediction models 162, and the trained therapeutic imagery effectiveness prediction models 162 are utilized to generate predicted imagery effectiveness data for the new therapeutic imagery. In some embodiments, the predicted imagery effectiveness data and the historical imagery effectiveness data are analyzed to select one or more effective therapeutic imagery attributes. Imagery definition data associated with the one or more effective therapeutic imagery attributes is then utilized to generate maximally effective therapeutic imagery. In one embodiment, maximally effective imagery definition data associated with the maximally effective therapeutic imagery is incorporated into historical imagery definition data for future use in administration to a patient. The above described system and process will be discussed in additional detail below with reference to the system of FIG. 3 and the process of FIG. 6.

FIG. 3 is a block diagram of a runtime environment 300 for utilizing trained therapeutic imagery effectiveness prediction models to generate generalized maximally effective therapeutic imagery, in accordance with one embodiment.

In various embodiments, runtime environment 300 includes application computing environment 301, average patient 302 and associated patient computing systems 304, software applications 315, health practitioner 312, and maximally effective therapeutic imagery 332. In one embodiment, runtime environment 300 further includes communications channel 309, which facilitates administration of maximally effective therapeutic imagery 332 to average patient 302, and communications channel 311, which facilitates retrieval of data from application computing environment 301. Application computing environment 301 includes therapeutic imagery database 102, which further includes historical therapeutic imagery data 104, such as historical imagery definition data 106, historical imagery media data 108, and historical imagery effectiveness data 109. In various embodiments, application computing environment 301 further includes additional data such as new therapeutic imagery test data 306, which further includes new imagery attribute 1 test data 308 through new imagery attribute n test data 310. In one embodiment, application computing environment 301 further includes imagery generation module 324, and imagery effectiveness threshold definition module 327. In various embodiments, imagery generation module 324 includes trained therapeutic imagery effectiveness prediction models 162, predicted therapeutic imagery effectiveness data 326, effective therapeutic imagery selection module 328, effective therapeutic imagery definition data 330, and maximally effective imagery generation module 331. In one embodiment, application computing environment 301 further includes maximally effective therapeutic imagery 332, and maximally effective imagery definition data 334. In one embodiment, application computing environment 301 further includes processor 336 and physical memory 338, which together coordinate the operation and interaction of the data and data processing modules associated with application computing environment 301. Each of the above listed elements will be discussed in further detail below.

In one embodiment, the previously trained therapeutic imagery effectiveness prediction models 162 are utilized in order to generate therapeutic imagery that will be maximally effective for average patients or for patients in general. In one embodiment, new therapeutic imagery test data 306 is generated or otherwise obtained, and is provided as input to the one or more trained therapeutic imagery effectiveness prediction models 162 of imagery generation module 324. In one embodiment, new therapeutic imagery test data 306 of FIG. 3 includes data representing any number of new therapeutic imagery attributes, which may be represented by new imagery attribute 1 test data 308 through new imagery attribute n test data 310. In one embodiment, once the new therapeutic imagery test data 306 has been provided as input to the one or more trained therapeutic imagery effectiveness prediction models 162 of imagery generation module 324, the one or more trained therapeutic imagery effectiveness prediction models 162 generate predicted therapeutic imagery effectiveness data 326. In various embodiments, predicted therapeutic imagery effectiveness data 326 represents the predicted effectiveness of each of the new therapeutic imagery attributes represented by new therapeutic imagery test data 306.

In one embodiment, once predicted therapeutic imagery effectiveness data 326 has been generated by trained therapeutic imagery effectiveness prediction models 162, it is passed to effective therapeutic imagery selection module 328 of imagery generation module 324 for further analysis. In one embodiment, effective therapeutic imagery selection module 328 selects one or more of the new therapeutic imagery attributes represented by new therapeutic imagery test data 306 that have been found to be effective. As discussed above, a determination as to what constitutes an “effective” imagery attribute may be made in any number of ways. As one illustrative example, imagery effectiveness threshold definition module 327 may set one or more threshold values for the effectiveness ratings represented by predicted therapeutic imagery effectiveness data 326. In one embodiment, imagery effectiveness threshold definition module 327 may be separate from imagery generation module 324. In one embodiment, imagery effectiveness threshold definition module 327 may be a sub-module of imagery generation module 324. In one embodiment, one or more threshold values may be explicitly set, for example, based on input from one or more health practitioners. In various other embodiments, imagery effectiveness threshold definition module 327 may derive or learn one or more threshold values based on analysis of training data, including, but not limited to historical imagery effectiveness data 109. In one embodiment, effective therapeutic imagery selection module 328 may also consider historical imagery effectiveness data 109 in determining and selecting effective imagery attributes.

In one embodiment, once effective therapeutic imagery selection module 328 has selected one or more effective therapeutic imagery attributes, effective therapeutic imagery definition data 330 is generated, which contains data defining the one or more selected effective imagery attributes. In one embodiment, maximally effective imagery generation module 331 utilizes effective therapeutic imagery definition data 330 to generate maximally effective therapeutic imagery 332. In various embodiments, maximally effective therapeutic imagery 332 may include any number and combination of maximally effective imagery attributes, and each of these imagery attributes or attribute combinations is defined by maximally effective imagery definition data 334.

Referring briefly to FIG. 1A and FIG. 3 together, in various embodiments, once maximally effective therapeutic imagery 332 has been generated by maximally effective imagery generation module 331 of imagery generation module 324, the maximally effective imagery definition data 334 representing the maximally effective therapeutic imagery 332 may be stored in a data structure, such as therapeutic imagery database 102, for further use. For example, in one embodiment, maximally effective imagery definition data 334 is incorporated into historical imagery definition data 106 of historical therapeutic imagery data 104, which, in some embodiments may be stored in therapeutic imagery database 102. As noted above, this is advantageous because it creates a feedback loop for the machine learning process, wherein the newly generated maximally effective therapeutic imagery 332 can be incorporated into the therapeutic imagery effectiveness model training data 158 of FIG. 1A, which is used to train the therapeutic imagery effectiveness prediction models 160. In this manner, the trained therapeutic imagery effectiveness prediction models 162 may be continually updated and refined as new patient imagery response data 126 is received from patient 118.

In one embodiment, once maximally effective therapeutic imagery 332 has been generated by maximally effective imagery generation module 331 of imagery generation module 324, the maximally effective therapeutic imagery 332 may then be administered to a patient, such as average patient 302. In some embodiments, once generated, the maximally effective therapeutic imagery 332 may be administered directly to average patient 302. In some embodiments, health practitioner 312 may review the maximally effective therapeutic imagery 332 prior to administration to average patient 302. In some embodiments, the maximally effective therapeutic imagery 332 may be stored in a data structure, such as therapeutic imagery database 102, for further use, but might not be administered to average patient 302.

In various embodiments, the maximally effective therapeutic imagery 332 may be administered to average patient 302 using one or more communication mechanisms 309. In some embodiments, communication mechanisms 309 include health practitioner 312 conducting a physical in-person meeting with average patient 302 to verbally guide average patient 302 through the maximally effective therapeutic imagery 332. In other embodiments, communication mechanisms 309 include administering the maximally effective therapeutic imagery 332 to average patient 302 remotely, for example through a website, or through one or more software applications 315 that can be executed from patient computing systems 304. In one embodiment, maximally effective therapeutic imagery 332 may be administered to average patient 302 directly by health practitioner 312. In one embodiment, maximally effective therapeutic imagery 332 may be administered to average patient 302 remotely, without the direct involvement of health practitioner 312. For example, maximally effective therapeutic imagery 332 may be self-administered by average patient 302. In one embodiment, maximally effective therapeutic imagery 332 may also be administered to average patient 302 remotely with partial involvement of health practitioner 312.

In various embodiments, patient computing systems 304 may include, but are not limited to, a desktop computing system, a mobile computing system, a virtual reality computing system, a gaming computing system, a computing system that utilizes one or more Internet of Things (IoT) devices, or any combination thereof.

As discussed above, in one embodiment, generation of maximally effective therapeutic imagery may also be performed in real-time such that the therapeutic imagery is reactive to various events, such as, but not limited to, changes in a patient's response data, mental status, physical status, and/or other changes associated with the patient or patient data.

### Process

FIG. 4 is a flow chart of a process 400 for creating trained therapeutic imagery effectiveness prediction models, in accordance with one embodiment.

Process 400 begins at BEGIN 402 and process flow proceeds to 404. At 404, a historical therapeutic imagery is selected for administration to one or more patients.

In one embodiment, the one or more patients are patients who have been diagnosed with a medical condition, and a determination is made as to whether the one or more patients will benefit from receiving therapeutic imagery. In one embodiment, once a determination has been made that one or more patients are likely to benefit from receiving therapeutic imagery, historical therapeutic imagery is selected for administration to the one or more patients. In various embodiments, a determination may be made as to which historical therapeutic imagery to administer to the one or more patients. This determination is at the discretion of the health practitioner responsible for each patient's treatment, and the determination may be based on a wide variety of factors, such as, but not limited to, the patient's medical condition, the severity of the patient's symptoms, personal patient preferences, the patient's medical history, the patient's age group, the patient's sex, and the patient's ethnicity.

In one embodiment, once historical therapeutic imagery is selected for administration to one or more patients at 404, process flow proceeds to 406. In one embodiment, at 406, the selected historical therapeutic imagery is administered to the one or more patients.

In various embodiments, the historical therapeutic imagery may be administered to the one or more patients using one or more communication mechanisms. In some embodiments, communication mechanisms include a health practitioner conducting a physical in-person meeting with a patient to verbally guide the patient through the historical therapeutic imagery. In other embodiments, communication mechanisms include administering the therapeutic imagery to the patient remotely, for example through a website, or through one or more software applications that can be executed from patient computing systems. In various embodiments, patient computing systems may include, but are not limited to, a desktop computing system, a mobile computing system, a virtual reality computing system, a gaming computing system, a computing system that utilizes one or more Internet of Things (IoT) devices, and/or any other type of computing system discussed herein, known at the time of filing, developed/made available after the time of filing, or any combination thereof.

In one embodiment, once the selected therapeutic imagery is administered to the one or more patients at 406, process flow proceeds to 408. In one embodiment, at 408, the patient responses to the historical therapeutic imagery are monitored to obtain patient imagery response data.

In various embodiments, patient imagery response data may include direct verbal or written feedback, and/or indirect feedback, such as an indication of whether a particular therapeutic imagery appears to be having an effect on the patient. The patient imagery response data may further include any other measureable data such as, but not limited to, click-stream data showing details related to patient engagement with the therapeutic imagery, for instance, the time that the patient spends engaging with the therapeutic imagery. The patient imagery response data may also include data received from devices such as, but not limited to, sleep trackers, or other types of physiological sensors that may be used to measure a patient's physiological state, such as heart rate, respiratory rate, and/or blood pressure. The patient imagery response data may further include data such as facial expressions, eye movement, and/or other measurable types of physical movement.

In one embodiment, once patient imagery response data is obtained at 408, process flow proceeds to 410. In one embodiment, at 410, the patient imagery response data is analyzed to determine the effectiveness of the historical therapeutic imagery for the one or more patients.

In various embodiments, effectiveness of therapeutic imagery for the one or more patients may be defined and determined in a variety of ways based on the patient imagery response data. For example, in practice, patient imagery response data is typically collected in a structured manner using established clinical procedures to ensure the validity of the data interpretation. In various embodiments, the results of the data interpretation may sometimes be referred to as “clinically validated outcome measures,” which may typically be defined as tools that are used in clinical settings to assess the current status of a patient. With respect to the embodiments disclosed herein, analysis of clinically validated outcome measures for a patient can help to determine therapeutic imagery effectiveness.

In one embodiment, once effectiveness of the historical therapeutic imagery is determined for the one or more patients at 410, process flow proceeds to 412. In one embodiment, at 412, patient imagery effectiveness data is generated representing the effectiveness of the historical therapeutic imagery for the one or more patients.

As detailed in the system discussion above, in various embodiments, at 412, effectiveness ratings are assigned to one or more therapeutic imagery attributes or combinations of imagery attributes, and the resulting imagery effectiveness data may be stored in one or more data structures for further use.

In one embodiment, once patient imagery effectiveness data is generated at 412, process flow proceeds to 414. In one embodiment, at 414, the patient imagery effectiveness data and patient data associated with the patient are analyzed to generate one or more patient profiles.

As discussed above, although one particular therapeutic imagery attribute or combination of imagery attributes may be effective for the general patient population, or for the average patient, the same imagery attributes may not be effective at all for a particular patient, or a particular type of patient, and as such, the effectiveness ratings of various imagery attributes are likely to vary significantly depending on the characteristics of the particular patient. It follows then, that in order to train one or more therapeutic imagery effectiveness prediction models, model training data that accounts for differences in imagery attribute effectiveness among different types of patients must be gathered and assembled. In various embodiments, the system and method disclosed herein builds a plurality of patient profiles based on known or obtained patient data. Patient data may include, for example, patient characteristics such as, but not limited to age, sex, ethnicity, religion, marital status, income level, geographic location, personal and family medical history, including the current medical issue that the therapy is designed to treat. In one embodiment, patient data may also include patient preference data, which in some embodiments, may be tied to preferences for specific therapeutic imagery attributes.

In one embodiment, patient imagery effectiveness data is correlated with specific profiles in the plurality of generated patient profiles, and the patient profile data may be stored in one or more data structures for later use. Any number of patient profiles may be generated, and the patient profiles are typically characterized by a combination of patient characteristics represented by the known or obtained patient data.

In various embodiments, the patient imagery effectiveness data represents a measure of how effective particular historical therapeutic imagery is for patients of that particular type. In one embodiment, patient imagery effectiveness data may include a list of hundreds, thousands, or millions of therapeutic imagery attributes and combinations of attributes, each with corresponding data indicating an effectiveness rating for each imagery attribute or combination of attributes. In some embodiments, an effectiveness rating for a therapeutic imagery attribute among a particular type of patient may be a single number representing an average of the effectiveness ratings for that therapeutic imagery attribute across all members of the group of patients defined by the patient profile type. In some embodiments an effectiveness rating for a therapeutic imagery attribute among a particular type of patient may be a range of numbers representing the effectiveness ratings for that therapeutic imagery attribute across all members of the group of patients defined by the patient profile type. In various other embodiments, a weighting system might be utilized, for instance to give higher weight to effectiveness ratings that are more common than others.

In one embodiment, once one or more patient profiles are generated at 414, process flow proceeds to 416. In one embodiment, at 416, historical therapeutic imagery data associated with the historical therapeutic imagery is correlated with patient profile data associated with the one or more patient profiles to generate therapeutic imagery effectiveness model training data.

In various embodiments, once a plurality of patient profiles are generated and associated with patient imagery effectiveness data, patient profile data and historical therapeutic imagery data is collected and the data is correlated to prepare it for transformation into therapeutic imagery effectiveness model training data for training one or more therapeutic imagery effectiveness models.

In one embodiment, once therapeutic imagery effectiveness model training data is generated at 416, process flow proceeds to 418. In one embodiment, at 418, the therapeutic imagery effectiveness model training data is used to train one or more machine learning based therapeutic imagery effectiveness prediction models, thereby resulting in the creation of one or more trained therapeutic imagery effectiveness prediction models.

In various embodiments, and largely depending on the machine learning based models used, the patient profile data and/or the historical therapeutic imagery data is processed using various methods know in the machine learning arts to identify elements and to vectorize the patient profile data and/or the historical therapeutic imagery data. As a specific illustrative example, in a case where the machine leaning based model is a supervised model, the historical therapeutic imagery data and the patient profile data can be analyzed and processed to identify individual elements found to be indicative of imagery effectiveness among certain types of patients, or among a generalized population of patients. These individual elements are then used to create therapeutic imagery effectiveness data vectors in multidimensional space, resulting in therapeutic imagery effectiveness model training data. The therapeutic imagery effectiveness model training data is then used as input data for training one or more therapeutic imagery effectiveness prediction models. The patient imagery effectiveness data for a patient profile that correlates with the therapeutic imagery effectiveness data vector associated with that patient profile is then used as a label for the resulting vector. In various embodiments, this process is repeated for each therapeutic imagery attribute defined by the historical imagery definition data, and for each patient profile type represented by the patient profile data. The result is that multiple, often millions, of correlated pairs of therapeutic imagery effectiveness data vectors and patient profiles are used to train the therapeutic imagery effectiveness prediction models. Consequently, this process results in the creation of one or more trained therapeutic imagery effectiveness prediction models.

In one embodiment, once one or more trained therapeutic imagery effectiveness prediction models are created at 418, process flow proceeds to 420. In one embodiment, at 420, a determination is made as to whether the one or more therapeutic imagery effectiveness prediction models should continue to be trained. In various embodiments, this determination may be made at the discretion of an operator or administrator of the system and method disclosed herein.

In one embodiment, upon a determination at 420 that the one or more therapeutic imagery effectiveness prediction models should continue to be trained, process flow returns to 404, and the above described operations may be repeated indefinitely.

In one embodiment, upon a determination at 420 that the one or more therapeutic imagery effectiveness prediction models should not continue to be trained, process flow proceeds to END 422 and the process 400 for creating trained therapeutic imagery effectiveness prediction models is exited to await new data and/or instructions.

FIG. 5 is a flow chart of a process 500 for utilizing trained therapeutic imagery effectiveness prediction models to generate maximally effective therapeutic imagery for a specific patient, in accordance with one embodiment.

Process 500 begins at BEGIN 502 and process flow proceeds to 504. In one embodiment, at 504, current patient data associated with a current patient and patient profile data associated with one or more predefined patient profiles are analyzed to select a patient profile that is the best match for the current patient.

In one embodiment, the current patient data is obtained, either directly from the current patient, from medical files associated with current patient, and/or current patient data may be retrieved from a database of previously collected patient data. In some embodiments, there may be no specific current patient, and test data may be used in place of current patient data. For example, a theoretical patient may be contemplated, and data describing characteristics of the theoretical patient may be used as test data in place of current patient data to generate maximally effective therapeutic imagery for the theoretical patient. In various embodiments, test data may be generated by one or more machine learning models that have been trained to predict effectiveness of the test data.

In various embodiments, once the current patient data has been obtained for the current patient, the current patient data is analyzed along with the patient profile data, in order to select a patient profile that most closely matches the characteristics of the current patient. In one embodiment, various characteristics of the current patient are compared to patient characteristics represented by the one or more patient profiles. As will be noted by those of skill in the art, various mechanisms and algorithms may be utilized to determine similarities between the current patient and the patient profiles represented by patient profile data. Similarity of the current patient to a particular patient profile may be determined by any number of factors, such as, but not limited to the current patient's age, sex, ethnicity, religion, marital status, income level, geographic location, personal and family medical history, including the current medical issue that the therapy is designed to treat. In one embodiment, one or more thresholds may be defined to determine how close of a match the current patient is to a particular patient profile.

In one embodiment, once a patient profile is selected at 504, process flow proceeds to 506. In one embodiment, at 506, the selected patient profile data is provided as input to one or more trained therapeutic imagery effectiveness prediction models.

In one embodiment, once the patient profile data is provided to one or more trained therapeutic imagery effectiveness prediction models at 506, process flow proceeds to 508. In one embodiment, at 508, new therapeutic imagery test data is generated representing one or more new therapeutic imagery attributes associated with therapeutic imagery.

In various embodiments, new therapeutic imagery test data is generated representing one or more new therapeutic imagery attributes. As noted above, in various embodiments, new therapeutic imagery may include potential therapeutic imagery or candidate therapeutic imagery, in the sense that it is imagery that is being considered for administration to one or more patients. In one embodiment, the new therapeutic imagery test data includes data representing any number of new therapeutic imagery attributes.

In one embodiment, once new therapeutic imagery test data is generated at 508, process flow proceeds to 510. In one embodiment, at 510, the new therapeutic imagery test data is provided as input to the one or more trained therapeutic imagery effectiveness prediction models.

In one embodiment, once the new therapeutic imagery test data is provided as input to the one or more trained therapeutic imagery effectiveness prediction models at 510, process flow proceeds to 512. In one embodiment, at 512, the one or more trained therapeutic imagery effectiveness prediction models are utilized to generate predicted therapeutic imagery effectiveness data for the new therapeutic imagery attributes represented by the new therapeutic imagery test data.

In various embodiments, the predicted therapeutic imagery effectiveness data represents the predicted effectiveness of each of the new therapeutic imagery attributes represented by the new therapeutic imagery test data for a patient who matches the patient profile type represented by the selected patient profile data.

In one embodiment, once predicted therapeutic imagery effectiveness data is generated at 512, process flow proceeds to 514. In one embodiment, at 514, the predicted therapeutic imagery effectiveness data associated with the new therapeutic imagery attributes and historical therapeutic imagery effectiveness data associated with historical therapeutic imagery attributes are analyzed to determine and select one or more effective therapeutic imagery attributes.

In one embodiment, one or more of the new therapeutic imagery attributes represented by the new therapeutic imagery test data are selected, wherein the new therapeutic imagery attributes have been found to be effective. As discussed above, a determination as to what constitutes an “effective” imagery attribute may be made by setting a threshold value for the effectiveness ratings represented by the predicted therapeutic imagery effectiveness data. In one embodiment, historical therapeutic imagery effectiveness data may also be considered in determining and selecting effective therapeutic imagery attributes.

In one embodiment, once one or more effective therapeutic imagery attributes are selected at 514, process flow proceeds to 516. In one embodiment, at 516, the one or more effective therapeutic imagery attributes are utilized to generate maximally effective therapeutic imagery.

In one embodiment, once one or more effective therapeutic imagery attributes have been selected, effective therapeutic imagery definition data is generated, which contains data defining the one or more selected effective imagery attributes. In one embodiment, the effective therapeutic imagery definition data is then used to generate maximally effective therapeutic imagery. In various embodiments, the maximally effective therapeutic imagery may include any number and combination of effective therapeutic imagery attributes, and each of these effective therapeutic imagery attributes or imagery attribute combinations is defined by maximally effective therapeutic imagery definition data.

In one embodiment, once maximally effective therapeutic imagery is generated at 516, process flow proceeds to 518. In one embodiment, at 518, maximally effective imagery definition data associated with the maximally effective therapeutic imagery is incorporated into historical imagery definition data for future use in administration to one or more patients.

Referring briefly to FIG. 4 and FIG. 5 together, in various embodiments, once maximally effective therapeutic imagery has been generated, the maximally effective imagery definition data representing the maximally effective therapeutic imagery may be stored in a data structure for further use. For example, in one embodiment, maximally effective imagery definition data is incorporated into historical imagery definition data, which, in some embodiments may be stored in a therapeutic imagery database. This is advantageous because it creates a feedback loop for the machine learning process, wherein the newly generated maximally effective therapeutic imagery can be incorporated into the therapeutic imagery effectiveness model training data, which is generated at 416 of FIG. 4, and is used to train the therapeutic imagery effectiveness prediction models at 418 of FIG. 4. In this manner, the trained therapeutic imagery effectiveness prediction models may be continually updated and refined as new patient imagery response data is received from one or more patients.

In one embodiment, once the maximally effective imagery definition data is incorporated into historical imagery definition data at 518, process flow proceeds to 520. In one embodiment, at 520, the maximally effective therapeutic imagery is administered to the current patient.

In one embodiment, once maximally effective therapeutic imagery has been generated, the maximally effective therapeutic imagery may then be administered to a patient, such as the current patient. In some embodiments, once generated, the maximally effective therapeutic imagery may be administered directly to the current patient. In some embodiments, a health practitioner may review the maximally effective therapeutic imagery prior to administration to the current patient. In some embodiments, the maximally effective therapeutic imagery may not be administered to the current patient and/or the maximally effective therapeutic imagery may be administered to a patient other than the current patient. In various embodiments, the therapeutic imagery may be administered to the current patient using one or more communication mechanisms. In some embodiments, communication mechanisms include a health practitioner conducting a physical in-person meeting with the current patient to verbally guide the current patient through the therapeutic imagery. In other embodiments, communication mechanisms include administering the therapeutic imagery to the current patient remotely, for example through a website, or through one or more software applications that can be executed from computing systems associated with the current patient.

In one embodiment, the maximally effective therapeutic imagery may be administered to a current patient (or a patient other than the current patient), and patient imagery response data may be collected in real-time such that new maximally effective therapeutic imagery may be generated and administered to current patient 202 in real-time, in reaction to changes in the current patient 202's response data, mental status, physical status, and/or other changes in associated current patient data.

In one embodiment, once the therapeutic imagery is administered to the patient at 520, process flow proceeds to END 522 and the process 500 for utilizing trained therapeutic imagery effectiveness prediction models to generate maximally effective therapeutic imagery for a specific patient is exited to await new data and/or instructions.

FIG. 6 is a flow chart of a process 600 for utilizing trained therapeutic imagery effectiveness prediction models to generate generalized maximally effective therapeutic imagery, in accordance with one embodiment.

Process 600 begins at BEGIN 602 and process flow proceeds to 604. In one embodiment, at 604, new therapeutic imagery test data is generated representing one or more new therapeutic imagery attributes associated with therapeutic imagery.

In various embodiments, new therapeutic imagery test data is generated representing one or more new therapeutic imagery attributes. As noted above, in various embodiments, new therapeutic imagery may include potential therapeutic imagery or candidate therapeutic imagery, in the sense that it is imagery that is being considered for administration to one or more patients. In one embodiment, the new therapeutic imagery test data includes data representing any number of new therapeutic imagery attributes.

In one embodiment, once new therapeutic imagery test data is generated at 604, process flow proceeds to 606. In one embodiment, at 606, the new therapeutic imagery test data is provided as input to the one or more trained therapeutic imagery effectiveness prediction models.

In one embodiment, once the new therapeutic imagery test data is provided as input to the one or more trained therapeutic imagery effectiveness prediction models at 606, process flow proceeds to 608. In one embodiment, at 608, the one or more trained therapeutic imagery effectiveness prediction models are utilized to generate predicted therapeutic imagery effectiveness data for the new therapeutic imagery attributes represented by the new therapeutic imagery test data.

In various embodiments, the predicted therapeutic imagery effectiveness data represents the predicted effectiveness for an average patient of each of the new therapeutic imagery attributes represented by the new therapeutic imagery test data.

In one embodiment, once predicted therapeutic imagery effectiveness data is generated at 608, process flow proceeds to 610. In one embodiment, at 610, the predicted therapeutic imagery effectiveness data associated with the new therapeutic imagery and historical therapeutic imagery effectiveness data associated with historical therapeutic imagery are analyzed to determine and select one or more effective therapeutic imagery attributes.

In one embodiment, one or more of the new therapeutic imagery attributes represented by the new therapeutic imagery test data are selected, wherein the new therapeutic imagery attributes have been found to be effective. As discussed above, a determination as to what constitutes an “effective” imagery attribute may be made by setting a threshold value for the effectiveness ratings represented by the predicted therapeutic imagery effectiveness data. In one embodiment, historical therapeutic imagery effectiveness data may also be considered in determining and selecting effective therapeutic imagery attributes.

In one embodiment, once one or more effective therapeutic imagery attributes are selected at 610, process flow proceeds to 612. In one embodiment, at 612, effective therapeutic imagery definition data associated with one or more effective therapeutic imagery attributes are utilized to generate maximally effective therapeutic imagery.

In one embodiment, once one or more effective therapeutic imagery attributes have been selected, effective therapeutic imagery definition data is generated, which contains data defining the one or more selected effective imagery attributes. In one embodiment, the effective therapeutic imagery definition data is then used to generate maximally effective therapeutic imagery. In various embodiments, the maximally effective therapeutic imagery may include any number and combination of effective therapeutic imagery attributes, and each of these effective therapeutic imagery attributes or imagery attribute combinations is defined by maximally effective therapeutic imagery definition data.

In one embodiment, once maximally effective therapeutic imagery is generated at 612, process flow proceeds to 614. In one embodiment, at 614, maximally effective imagery definition data associated with the maximally effective therapeutic imagery is incorporated into historical imagery definition data for future use in administration to one or more patients.

Referring briefly to FIG. 4 and FIG. 6 together, in various embodiments, once maximally effective therapeutic imagery has been generated, the maximally effective imagery definition data representing the maximally effective therapeutic imagery may be stored in a data structure for further use. For example, in one embodiment, maximally effective imagery definition data is incorporated into historical imagery definition data, which, in some embodiments may be stored in a therapeutic imagery database. This is advantageous because it creates a feedback loop for the machine learning process, wherein the newly generated maximally effective therapeutic imagery can be incorporated into the therapeutic imagery effectiveness model training data, which is generated at 416 of FIG. 4, and is used to train the therapeutic imagery effectiveness prediction models at 418 of FIG. 4. In this manner, the trained therapeutic imagery effectiveness prediction models may be continually updated and refined as new patient imagery response data is received from one or more patients.

In one embodiment, once the maximally effective imagery definition data is incorporated into historical imagery definition data at 614, process flow proceeds to 616. In one embodiment, at 616, the maximally effective therapeutic imagery is administered to an average patient.

In one embodiment, once maximally effective therapeutic imagery has been generated, the maximally effective therapeutic imagery may then be administered to a patient, such as an average patient. In some embodiments, once generated, the maximally effective therapeutic imagery may be administered directly to the average patient. In some embodiments, a health practitioner may review the maximally effective therapeutic imagery prior to administration to the average patient. In some embodiments, the maximally effective therapeutic imagery may not be administered to an average patient. In various embodiments, the therapeutic imagery may be administered to the average patient using one or more communication mechanisms. In some embodiments, communication mechanisms include a health practitioner conducting a physical in-person meeting with the average patient to verbally guide the average patient through the therapeutic imagery. In other embodiments, communication mechanisms include administering the therapeutic imagery to the average patient remotely, for example through a website, or through one or more software applications that can be executed from computing systems associated with the current patient.

In one embodiment, once the maximally effective imagery definition data is administered to an average patient at 616, process flow proceeds to END 618 and the process 600 for utilizing trained therapeutic imagery effectiveness prediction models to generate generalized maximally effective therapeutic imagery is exited to await new data and/or instructions.

In one embodiment, a computing system implemented method comprises administering historical therapeutic imagery to one or more patients, and analyzing patient imagery response data representing the responses of the one or more patients to the historical therapeutic imagery to determine the effectiveness of the historical therapeutic imagery for the one or more patients. In one embodiment the method further comprises analyzing patient imagery effectiveness data representing the effectiveness of the historical therapeutic imagery for the one or more patients and patient data associated with the one or more patients to generate one or more patient profiles, and correlating historical therapeutic imagery data associated with the historical therapeutic imagery, the patient imagery effectiveness data, and patient profile data associated with the one or more patient profiles to generate therapeutic imagery effectiveness model training data. In one embodiment, the method further comprises utilizing the therapeutic imagery effectiveness model training data to train one or more therapeutic imagery effectiveness prediction models, thereby resulting in the creation of one or more trained therapeutic imagery effectiveness prediction models, and utilizing the one or more trained therapeutic imagery effectiveness prediction models to generate maximally effective therapeutic imagery.

In one embodiment, the historical therapeutic imagery is administered to the one or more patients remotely. In one embodiment, the responses of the one or more patients to the historical therapeutic imagery are monitored remotely. In one embodiment, the historical therapeutic imagery is administered to the one or more patients as part of a cognitive behavioral therapy (CBT) treatment used to treat patients diagnosed with irritable bowel syndrome (IBS).

In one embodiment, utilizing the one or more trained therapeutic imagery effectiveness prediction models to generate maximally effective therapeutic imagery further includes analyzing current patient data associated with a current patient and patient profile data associated with one or more predefined patient profiles to select a patient profile for the current patient and providing selected patient profile data associated with the selected patient profile to the one or more trained therapeutic imagery effectiveness prediction models. In one embodiment, utilizing the one or more trained therapeutic imagery effectiveness prediction models to generate maximally effective therapeutic imagery further includes generating new therapeutic imagery test data representing one or more new therapeutic imagery attributes associated with therapeutic imagery and providing the new therapeutic imagery test data to the one or more trained therapeutic imagery effectiveness prediction models. In one embodiment, utilizing the one or more trained therapeutic imagery effectiveness prediction models to generate maximally effective therapeutic imagery further includes utilizing the one or more trained therapeutic imagery effectiveness prediction models to generate predicted imagery effectiveness data for the new therapeutic imagery represented by the new therapeutic imagery test data, analyzing the predicted therapeutic imagery effectiveness data associated with the new therapeutic imagery and historical imagery effectiveness data associated with the historical therapeutic imagery to determine and select one or more effective therapeutic imagery attributes, utilizing effective therapeutic imagery definition data associated with the one or more effective therapeutic imagery attributes to generate maximally effective therapeutic imagery, and upon generation of maximally effective therapeutic imagery, taking one or more actions.

In one embodiment, generating maximally effective therapeutic imagery includes replacing one or more of the historical therapeutic imagery attributes with one or more of the effective therapeutic imagery attributes. In one embodiment, taking one or more actions includes one or more of storing maximally effective therapeutic imagery definition data associated with the maximally effective therapeutic imagery for use in administration to a patient, administering the maximally effective therapeutic imagery to a patient, and incorporating maximally effective therapeutic imagery data associated with the maximally effective therapeutic imagery into the therapeutic imagery effectiveness model training data.

In one embodiment, administering the maximally effective therapeutic imagery to a patient further includes performing continuously, for a defined duration of time, one or more of monitoring the responses of the patient to the maximally effective therapeutic imagery in real-time to obtain real-time patient imagery response data, updating the one or more trained therapeutic imagery effectiveness prediction models as new therapeutic imagery effectiveness model training data is received, utilizing the real-time patient imagery response data and the updated trained therapeutic imagery effectiveness prediction models to generate new maximally effective therapeutic imagery, and administering the new maximally effective therapeutic imagery to the patient.

In one embodiment, the maximally effective therapeutic imagery is administered to a patient remotely. In one embodiment, the maximally effective therapeutic imagery is administered to a patient as part of a cognitive behavioral therapy (CBT) treatment used to treat patients diagnosed with irritable bowel syndrome (IBS). In one embodiment, the one or more therapeutic imagery effectiveness prediction models are machine learning based models that are one or more of supervised machine learning-based models, semi supervised machine learning-based models, unsupervised machine learning-based models, classification machine learning-based models, logistical regression machine learning-based models, neural network machine learning-based models, and deep learning machine learning-based models.

In one embodiment, a system comprises one or more processors and one or more physical memories, the one or more physical memories having stored therein data representing instructions, which when processed by the one or more processors perform the above described computer implemented method/process.

The above described method and system result in generation of maximally effective therapeutic imagery, which may be administered to one or more patients, thus ensuring that the patients receive effective care, support, and treatment. Further, the machine learning processes described above employs a feedback loop, such that the one or more therapeutic imagery effectiveness prediction models can be dynamically refined to account for newly received effectiveness data, thus continually improving the accuracy of future therapeutic imagery effectiveness predictions generated by the model. Additionally, the above described method and system enables maximally effective therapeutic imagery to be reactive to real-time changes associated with a patient. As a result of these and other disclosed features, discussed in detail above, the disclosed embodiments provide an effective and efficient technical solution to the technical problem of dynamically generating therapeutic imagery to ensure that patients receive maximally effective care, support, and treatment.

Consequently, the embodiments disclosed herein are not an abstract idea, and are well-suited to a wide variety of practical applications. Further, many of the embodiments disclosed herein require processing and analysis of billions of data points and combinations of data points, and thus, the technical solution disclosed herein cannot be implemented solely by mental steps or pen and paper, is not an abstract idea, and is, in fact, directed to providing technical solutions to long-standing technical problems associated with predicting the effectiveness of therapeutic imagery and dynamically generating therapeutic imagery that will be maximally effective when administered to a patient.

Additionally, the disclosed method and system for dynamically generating therapeutic imagery using machine learning models requires a specific process comprising the aggregation and detailed analysis of large quantities of patient data, therapeutic imagery data, and therapeutic imagery effectiveness data, and as such, does not encompass, embody, or preclude other forms of innovation in the area of therapeutic imagery generation. Further, the disclosed embodiments of systems and methods for dynamically generating therapeutic imagery using machine learning models are not abstract ideas for at least several reasons.

First, dynamically generating therapeutic imagery using machine learning models is not an abstract idea because it is not merely an idea in and of itself. For example, the process cannot be performed mentally or using pen and paper, as it is not possible for the human mind to identify, process, and analyze the millions of possible patient characteristics, therapeutic imagery attributes, combinations of imagery attributes, and associated therapeutic imagery effectiveness data, even with pen and paper to assist the human mind and even with unlimited time.

Second, dynamically generating therapeutic imagery using machine learning models is not a fundamental economic practice (e.g., is not merely creating a contractual relationship, hedging, mitigating a settlement risk, etc.).

Third, dynamically generating therapeutic imagery using machine learning models is not merely a method of organizing human activity (e.g., managing a game of bingo). Rather, in the disclosed embodiments, the method and system for dynamically generating therapeutic imagery using machine learning models provides a tool that significantly improves the fields of medical and mental health care. Through the disclosed embodiments, health practitioners are provided with a tool to help them generate improved therapeutic imagery, which ensures that patients are provided with personalized and maximally effective assistance, treatment, and care. As such, the method and system disclosed herein is not an abstract idea, and also serves to integrate the ideas disclosed herein into practical applications of those ideas.

Fourth, although mathematics may be used to implement the embodiments disclosed herein, the systems and methods disclosed and claimed herein are not abstract ideas because the disclosed systems and methods are not simply a mathematical relationship/formula.

It should be noted that the language used in the specification has been principally selected for readability, clarity, and instructional purposes, and may not have been selected to delineate or circumscribe the inventive subject matter. Accordingly, the disclosure of the present invention is intended to be illustrative, but not limiting, of the scope of the invention, which is set forth in the claims below.

The present invention has been described in particular detail with respect to specific possible embodiments. Those of skill in the art will appreciate that the invention may be practiced in other embodiments. For example, the nomenclature used for components, capitalization of component designations and terms, the attributes, data structures, or any other programming or structural aspect is not significant, mandatory, or limiting, and the mechanisms that implement the invention or its features can have various different names, formats, or protocols. Further, the system or functionality of the invention may be implemented via various combinations of software and hardware, as described, or entirely in hardware elements. Also, particular divisions of functionality between the various components described herein are merely exemplary, and not mandatory or significant. Consequently, functions performed by a single component may, in other embodiments, be performed by multiple components, and functions performed by multiple components may, in other embodiments, be performed by a single component.

In the discussion above, certain aspects of one embodiment include process steps and/or operations and/or instructions described herein for illustrative purposes in a particular order and/or grouping. However, the particular order and/or grouping shown and discussed herein are illustrative only and not limiting. Those of ordinary skill in the art will recognize that other orders and/or grouping of the process steps and/or operations and/or instructions are possible and, in some embodiments, one or more of the process steps and/or operations and/or instructions discussed above can be combined and/or deleted. In addition, portions of one or more of the process steps and/or operations and/or instructions can be re-grouped as portions of one or more other of the process steps and/or operations and/or instructions discussed herein. Consequently, the particular order and/or grouping of the process steps and/or operations and/or instructions discussed herein do not limit the scope of the invention as claimed below.

As discussed in more detail above, using the above embodiments, with little or no modification and/or input, there is considerable flexibility, adaptability, and opportunity for customization to meet the specific needs of various parties under numerous circumstances.

Some portions of the above description present the features of the present invention in terms of algorithms and symbolic representations of operations, or algorithm-like representations, of operations on information/data. These algorithmic or algorithm-like descriptions and representations are the means used by those of skill in the art to most effectively and efficiently convey the substance of their work to others of skill in the art. These operations, while described functionally or logically, are understood to be implemented by computer programs or computing systems. Furthermore, it has also proven convenient at times to refer to these arrangements of operations as steps or modules or by functional names, without loss of generality.

Unless specifically stated otherwise, as would be apparent from the above discussion, it is appreciated that throughout the above description, discussions utilizing terms such as, but not limited to, “activating”, “accessing”, “adding”, “aggregating”, “alerting”, “applying”, “analyzing”, “associating”, “calculating”, “capturing”, “categorizing”, “classifying”, “comparing”, “creating”, “defining”, “detecting”, “determining”, “distributing”, “eliminating”, “encrypting”, “extracting”, “filtering”, “forwarding”, “generating”, “identifying”, “implementing”, “informing”, “monitoring”, “obtaining”, “posting”, “processing”, “providing”, “receiving”, “requesting”, “saving”, “sending”, “storing”, “substituting”, “transferring”, “transforming”, “transmitting”, “using”, etc., refer to the action and process of a computing system or similar electronic device that manipulates and operates on data represented as physical (electronic) quantities within the computing system memories, resisters, caches or other information storage, transmission or display devices.

The present invention also relates to an apparatus or system for performing the operations described herein. This apparatus or system may be specifically constructed for the required purposes, or the apparatus or system can comprise a system selectively activated or configured/reconfigured by a computer program stored on a non-transitory computer readable medium for carrying out instructions using a processor to execute a process, as discussed or illustrated herein that can be accessed by a computing system or other device.

Those of ordinary skill in the art will readily recognize that the algorithms and operations presented herein are not inherently related to any particular computing system, computer architecture, computer or industry standard, or any other specific apparatus. Various systems may also be used with programs in accordance with the teaching herein, or it may prove more convenient/efficient to construct more specialized apparatuses to perform the required operations described herein. The required structure for a variety of these systems will be apparent to those of ordinary skill in the art, along with equivalent variations. In addition, the present invention is not described with reference to any particular programming language and it is appreciated that a variety of programming languages may be used to implement the teachings of the present invention as described herein, and any references to a specific language or languages are provided for illustrative purposes only and for enablement of the invention as contemplated by the inventors at the time of filing.

The present invention is well suited to a wide variety of computer network systems operating over numerous topologies. Within this field, the configuration and management of large networks comprise storage devices and computers that are communicatively coupled to similar or dissimilar computers and storage devices over a private network, a LAN, a WAN, a private network, or a public network, such as the Internet.

It should also be noted that the language used in the specification has been principally selected for readability, clarity and instructional purposes, and may not have been selected to delineate or circumscribe the inventive subject matter. Accordingly, the disclosure of the present invention is intended to be illustrative, but not limiting, of the scope of the invention, which is set forth in the claims below.

In addition, the operations shown in the figures, or as discussed herein, are identified using a particular nomenclature for ease of description and understanding, but other nomenclature is often used in the art to identify equivalent operations.

Therefore, numerous variations, whether explicitly provided for by the specification or implied by the specification or not, may be implemented by one of skill in the art in view of this disclosure.

What is claimed is:

1. A computing system implemented method comprising:
administering historical therapeutic imagery to one or more patients;
analyzing patient imagery response data representing the responses of the one or more patients to the historical therapeutic imagery to determine the effectiveness of the historical therapeutic imagery for the one or more patients;
analyzing patient imagery effectiveness data representing the effectiveness of the historical therapeutic imagery for the one or more patients and patient data associated with the one or more patients to generate one or more patient profiles;
correlating historical therapeutic imagery data associated with the historical therapeutic imagery, the patient imagery effectiveness data, and patient profile data associated with the one or more patient profiles to generate therapeutic imagery effectiveness model training data;
utilizing the therapeutic imagery effectiveness model training data to train one or more therapeutic imagery effectiveness prediction models, thereby resulting in the creation of one or more trained therapeutic imagery effectiveness prediction models; and
utilizing the one or more trained therapeutic imagery effectiveness prediction models to generate maximally effective therapeutic imagery.

2. The method of claim 1 wherein the historical therapeutic imagery is administered to the one or more patients remotely.

3. The method of claim 1 wherein the responses of the one or more patients to the historical therapeutic imagery are monitored remotely.

4. The method of claim 1 wherein utilizing the one or more trained therapeutic imagery effectiveness prediction models to generate maximally effective therapeutic imagery further includes:
analyzing current patient data associated with a current patient and patient profile data associated with one or more predefined patient profiles to select a patient profile for the current patient;
providing selected patient profile data associated with the selected patient profile to the one or more trained therapeutic imagery effectiveness prediction models;
generating new therapeutic imagery test data representing one or more new therapeutic imagery attributes associated with therapeutic imagery;
providing the new therapeutic imagery test data to the one or more trained therapeutic imagery effectiveness prediction models;
utilizing the one or more trained therapeutic imagery effectiveness prediction models to generate predicted imagery effectiveness data for the new therapeutic imagery represented by the new therapeutic imagery test data;
analyzing the predicted therapeutic imagery effectiveness data associated with the new therapeutic imagery and historical imagery effectiveness data associated with the historical therapeutic imagery to determine and select one or more effective therapeutic imagery attributes;
utilizing effective therapeutic imagery definition data associated with the one or more effective therapeutic imagery attributes to generate maximally effective therapeutic imagery; and
upon generation of maximally effective therapeutic imagery, taking one or more actions.

5. The method of claim 4 wherein generating maximally effective therapeutic imagery includes replacing one or more of the historical therapeutic imagery attributes with one or more of the effective therapeutic imagery attributes.

6. The method of claim 4 wherein taking one or more actions includes one or more of:
storing maximally effective therapeutic imagery definition data associated with the maximally effective therapeutic imagery for use in administration to a patient;
administering the maximally effective therapeutic imagery to a patient; and
incorporating maximally effective therapeutic imagery data associated with the maximally effective therapeutic imagery into the therapeutic imagery effectiveness model training data.

7. The method of claim 6 wherein administering the maximally effective therapeutic imagery to a patient further includes one or more of:
monitoring the responses of the patient to the maximally effective therapeutic imagery in real-time to obtain real-time patient imagery response data;
updating the one or more trained therapeutic imagery effectiveness prediction models as new therapeutic imagery effectiveness model training data is received;
utilizing the real-time patient imagery response data and the updated trained therapeutic imagery effectiveness prediction models to generate new maximally effective therapeutic imagery; and
administering the new maximally effective therapeutic imagery to the patient.

8. The method of claim 6 wherein the maximally effective therapeutic imagery is administered to a patient remotely.

9. The method of claim 6 wherein the maximally effective therapeutic imagery is administered to a patient as part of a cognitive behavioral therapy (CBT) treatment used to treat patients diagnosed with irritable bowel syndrome (IBS).

10. The method of claim 1 wherein the one or more therapeutic imagery effectiveness prediction models are machine learning based models that are one or more of:
supervised machine learning-based models;
semi supervised machine learning-based models;
unsupervised machine learning-based models;
classification machine learning-based models;
logistical regression machine learning-based models;
neural network machine learning-based models; and
deep learning machine learning-based models.

11. A system comprising:
one or more processors; and
one or more physical memories, the one or more physical memories having stored therein data representing instructions which when processed by the one or more processors perform a process, the process comprising:
administering historical therapeutic imagery to one or more patients;
analyzing patient imagery response data representing the responses of the one or more patients to the historical therapeutic imagery to determine the effectiveness of the historical therapeutic imagery for the one or more patients;
analyzing patient imagery effectiveness data representing the effectiveness of the historical therapeutic imagery for the one or more patients and patient data associated with the one or more patients to generate one or more patient profiles;
correlating historical therapeutic imagery data associated with the historical therapeutic imagery, the patient imagery effectiveness data, and patient profile data associated with the one or more patient profiles to generate therapeutic imagery effectiveness model training data;
utilizing the therapeutic imagery effectiveness model training data to train one or more therapeutic imagery effectiveness prediction models, thereby resulting in the creation of one or more trained therapeutic imagery effectiveness prediction models; and
utilizing the one or more trained therapeutic imagery effectiveness prediction models to generate maximally effective therapeutic imagery.

12. The system of claim 11 wherein utilizing the one or more trained therapeutic imagery effectiveness prediction models to generate maximally effective therapeutic imagery further includes:
analyzing current patient data associated with a current patient and patient profile data associated with one or more predefined patient profiles to select a patient profile for the current patient;
providing selected patient profile data associated with the selected patient profile to the one or more trained therapeutic imagery effectiveness prediction models;
generating new therapeutic imagery test data representing one or more new therapeutic imagery attributes associated with therapeutic imagery;
providing the new therapeutic imagery test data to the one or more trained therapeutic imagery effectiveness prediction models;
utilizing the one or more trained therapeutic imagery effectiveness prediction models to generate predicted imagery effectiveness data for the new therapeutic imagery represented by the new therapeutic imagery test data;
analyzing the predicted therapeutic imagery effectiveness data associated with the new therapeutic imagery and historical imagery effectiveness data associated with the historical therapeutic imagery to determine and select one or more effective therapeutic imagery attributes;
utilizing effective therapeutic imagery definition data associated with the one or more effective therapeutic imagery attributes to generate maximally effective therapeutic imagery; and
upon generation of maximally effective therapeutic imagery, taking one or more actions.

13. The system of claim 12 wherein generating maximally effective therapeutic imagery includes replacing one or more of the historical therapeutic imagery attributes with one or more of the effective therapeutic imagery attributes.

14. The system of claim 12 wherein taking one or more actions includes one or more of:
storing maximally effective therapeutic imagery definition data associated with the maximally effective therapeutic imagery for use in administration to a patient;
administering the maximally effective therapeutic imagery to a patient; and
incorporating maximally effective therapeutic imagery data associated with the maximally effective therapeutic imagery into the therapeutic imagery effectiveness model training data.

15. The system of claim 14 wherein administering the maximally effective therapeutic imagery to a patient further includes performing continuously, for a defined duration of time, one or more of:
monitoring the responses of the patient to the maximally effective therapeutic imagery in real-time to obtain real-time patient imagery response data;
updating the one or more trained therapeutic imagery effectiveness prediction models as new therapeutic imagery effectiveness model training data is received;
utilizing the real-time patient imagery response data and the updated trained therapeutic imagery effectiveness prediction models to generate new maximally effective therapeutic imagery; and
administering the new maximally effective therapeutic imagery to the patient.

16. The system of claim 14 wherein the maximally effective therapeutic imagery is administered to a patient as part of a cognitive behavioral therapy (CBT) treatment used to treat patients diagnosed with irritable bowel syndrome (IBS).

17. The system of claim 11 wherein the one or more therapeutic imagery effectiveness prediction models are machine learning based models that are one or more of:
supervised machine learning-based models;
semi supervised machine learning-based models;
unsupervised machine learning-based models;
classification machine learning-based models;
logistical regression machine learning-based models;
neural network machine learning-based models; and
deep learning machine learning-based models.

18. A computing system implemented method comprising:
selecting historical therapeutic imagery for administration to one or more patients;
administering the selected historical therapeutic imagery to the one or more patients;
monitoring the responses of the one or more patients to the historical therapeutic imagery to obtain patient imagery response data;
analyzing the patient imagery response data to determine the effectiveness of the historical therapeutic imagery for the one or more patients;
generating patient imagery effectiveness data representing the effectiveness of the historical therapeutic imagery for the one or more patients;
analyzing the patient imagery effectiveness data and patient data associated with the one or more patients to generate one or more patient profiles;
correlating historical therapeutic imagery data associated with the historical therapeutic imagery with patient profile data associated with the one or more patient profiles to generate therapeutic imagery effectiveness model training data;
utilizing the therapeutic imagery effectiveness model training data to train one or more therapeutic imagery effectiveness prediction models, thereby resulting in the creation of one or more trained therapeutic imagery effectiveness prediction models;
analyzing current patient data associated with a current patient and patient profile data associated with one or more predefined patient profiles to select a patient profile for the current patient;
providing selected patient profile data associated with the selected patient profile to the one or more trained therapeutic imagery effectiveness prediction models;
generating new therapeutic imagery test data representing one or more new therapeutic imagery attributes associated with the therapeutic imagery;
providing the new therapeutic imagery test data to the one or more trained therapeutic imagery effectiveness prediction models;
utilizing the one or more trained therapeutic imagery effectiveness prediction models to generate predicted therapeutic imagery effectiveness data for the new therapeutic imagery represented by the new therapeutic imagery test data;
analyzing the predicted therapeutic imagery effectiveness data associated with the new therapeutic imagery and historical imagery effectiveness data associated with the historical therapeutic imagery to determine and select one or more effective therapeutic imagery attributes;
utilizing effective therapeutic imagery definition data associated with the one or more effective therapeutic imagery attributes to generate maximally effective therapeutic imagery;
upon generation of maximally effective therapeutic imagery, taking one or more actions.

19. The method of claim 18 wherein taking one or more actions includes one or more of:
storing maximally effective therapeutic imagery definition data associated with the maximally effective therapeutic imagery for use in administration to a patient;
administering the maximally effective therapeutic imagery to a patient; and
incorporating maximally effective therapeutic imagery data associated with the maximally effective therapeutic imagery into the therapeutic imagery effectiveness model training data.

20. The method of claim 19 wherein administering the maximally effective therapeutic imagery to a patient further includes performing continuously, for a defined duration of time, one or more of:
monitoring the responses of the patient to the maximally effective therapeutic imagery in real-time to obtain real-time patient imagery response data;
updating the one or more trained therapeutic imagery effectiveness prediction models as new therapeutic imagery effectiveness model training data is received;
utilizing the real-time patient imagery response data and the updated trained therapeutic imagery effectiveness prediction models to generate new maximally effective therapeutic imagery; and
administering the new maximally effective therapeutic imagery to the patient.
